(19) United States
(12) Patent Application Publication
(10) Pub. No.: US 2025/0006328 A1
(43) Pub. Date: Jan. 2, 2025

(54) SYSTEM AND METHOD FOR REAL-TIME AUGMENTATION OF PROVIDER PROGRESS NOTES AND SELECTIVE RECOMMENDATION OF PATIENT DIAGNOSES TO A PROVIDER

(71) Applicant: Regard Technologies, Inc., Los Angeles, CA (US)

(72) Inventors: Eli Ben-Joseph, Los Angeles, CA (US); Nathaniel Douglas Wilson, Los Angeles, CA (US); Thomas William Moulia, Los Angeles, CA (US); Daniel Lee Tryon, Los Angeles, CA (US); Francisco Alvarez, Los Angeles, CA (US)

(21) Appl. No.: 18/756,869

(22) Filed: Jun. 27, 2024

Related U.S. Application Data
(60) Provisional application No. 63/524,495, filed on Jun. 30, 2023.
(60) Provisional application No. 63/524,508, filed on Jun. 30, 2023.
(60) Provisional application No. 63/636,019, filed on Apr. 18, 2024.

(51) Int. Cl. G16H 15/00; G16H 10/60

(52) U.S. Cl. CPC G16H 15/00; G16H 10/60

(57) ABSTRACT

One variation of a method includes: receiving identification of a diagnosis for an encounter with a patient via a provider portal accessed by a provider; accessing a health record for the patient; accessing a diagnostic model including a module corresponding to the diagnosis; extracting a set of patient indicators, supporting the diagnosis for the patient, from the health record; and transmitting a prompt to the provider, via the provider portal, to review each patient indicator for specifying in a provider note generated for the encounter. This variation of the method further includes, in response to receiving selection of a subset of patient indicators, for specifying in the provider note, from the provider: appending the provider note with the subset of patient indicators; predicting an acceptance score for the provider note; and, in response to the acceptance score exceeding a threshold score, verifying the provider note.

### CROSS-REFERENCE TO RELATED APPLICATIONS

This Application claims the benefit of U.S. Provisional Application No. 63/636,019, filed on 18 Apr. 2024, U.S. Provisional Application No. 63/524,508, filed on 30 Jun. 2023, and U.S. Provisional Application No. 63/524,495, filed on 30 Jun. 2023, all of which are incorporated in their entireties by this reference.

### TECHNICAL FIELD

This invention relates generally to the field of health care and more specifically to a new and useful system and method for real-time augmentation of provider notes with supporting evidence for a diagnosis in the field of health care.

### BRIEF DESCRIPTION OF THE FIGURES

FIG. 1 is a flowchart representation of a method.

FIG. 2 is a flowchart representation of one variation of the method;

FIG. 3 is a flowchart representation of one variation of the method;

FIG. 4 is a flowchart representation of one variation of the method;

FIG. 5 is a flowchart representation of one variation of the method;

FIG. 6 is a flowchart representation of one variation of the method;

FIG. 7 is a flowchart representation of one variation of the method;

FIG. 8 is a flowchart representation of one variation of the method;

FIG. 9 is a flowchart representation of one variation of the method;

FIG. 10 is a flowchart representation of one variation of the method;

FIG. 11 is a flowchart representation of one variation of the method; and

FIG. 12 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method: Live Diagnosis+Note Augmentation

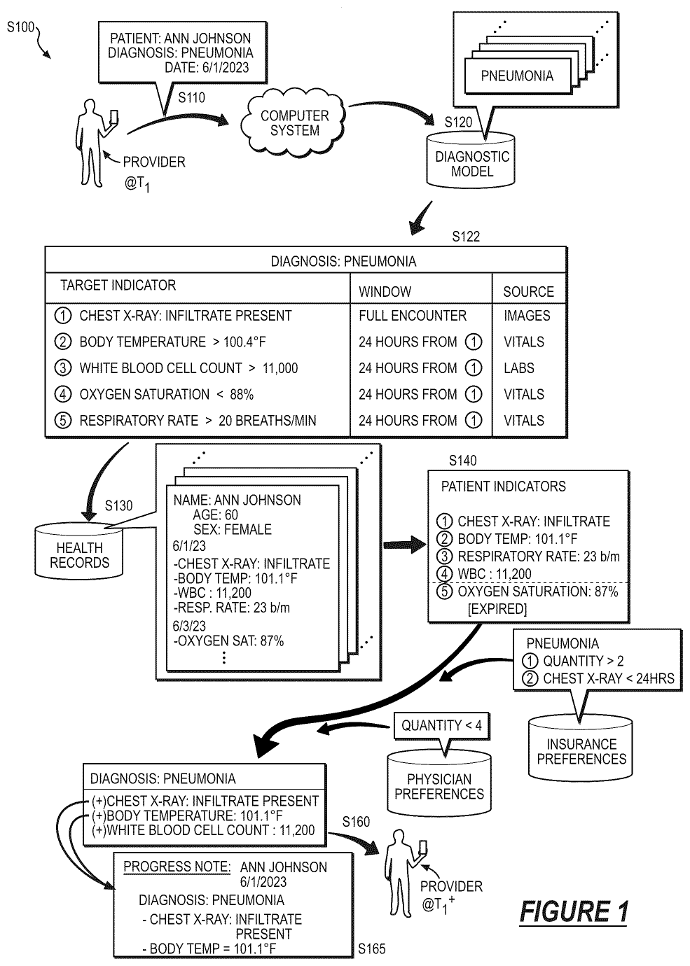

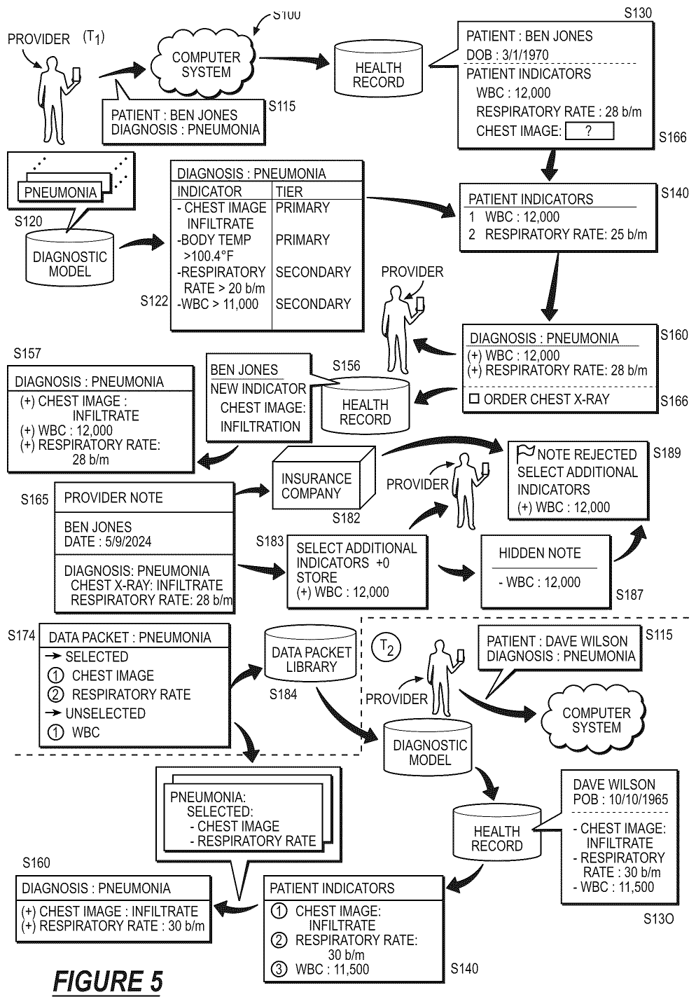

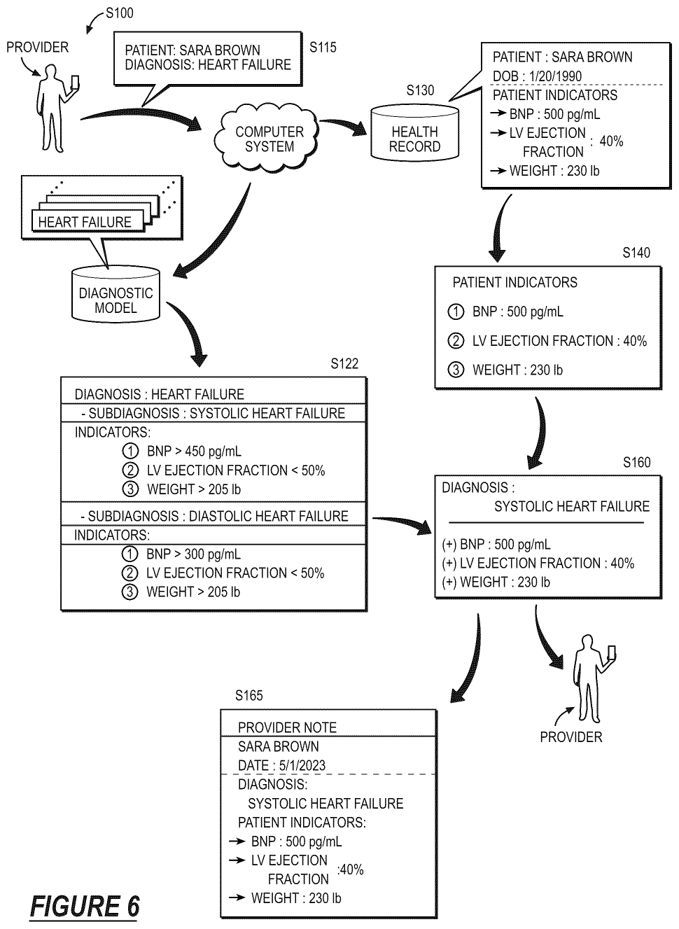

As shown in FIGS. 1 and 4-6, a method S100 includes: receiving identification of a first diagnosis, in a population of diagnoses, for a first encounter with a first patient from an instance of a provider portal accessed by a provider in Block S110; accessing a health record, in a population of health records, corresponding to the first patient and including a corpus of patient data associated with the first patient in Block S130; accessing a diagnostic model including a population of modules, corresponding to a population of diagnoses, each module, in the population of modules, defining a set of target indicators supporting a corresponding diagnosis in the population of diagnoses. The method S100 further includes, for a first module, in the population of modules, corresponding to the first diagnosis: accessing a first set of target indicators defined for the first diagnosis in the first module in Block S122; extracting a first set of patient indicators, from the corpus of patient data, corresponding to the first set of target indicators and supporting the first diagnosis for the first patient in Block S140; generating a first notification including the first set of patient indicators and a first prompt to review each patient indicator, in the first set of patient indicators, for specifying in the first provider note generated for the first encounter and indicating the first diagnosis for the first patient; and transmitting the first notification to the provider via the provider portal in Block S160. The method S100 further includes, in response to receiving selection of a first subset of patient indicators, in the first set of patient indicators, from the provider via the provider portal: appending the provider note with the first subset of patient indicators linked to the first diagnosis in Block S165; predicting a first acceptance score for the first provider note based on the first diagnosis and the first subset of patient indicators, the first acceptance score representing a likelihood of acceptance of the first diagnosis for the first patient during the first encounter represented by the first provider note in Block S170; and, in response to the first acceptance score exceeding a threshold score, verifying the first provider note in Block S180.

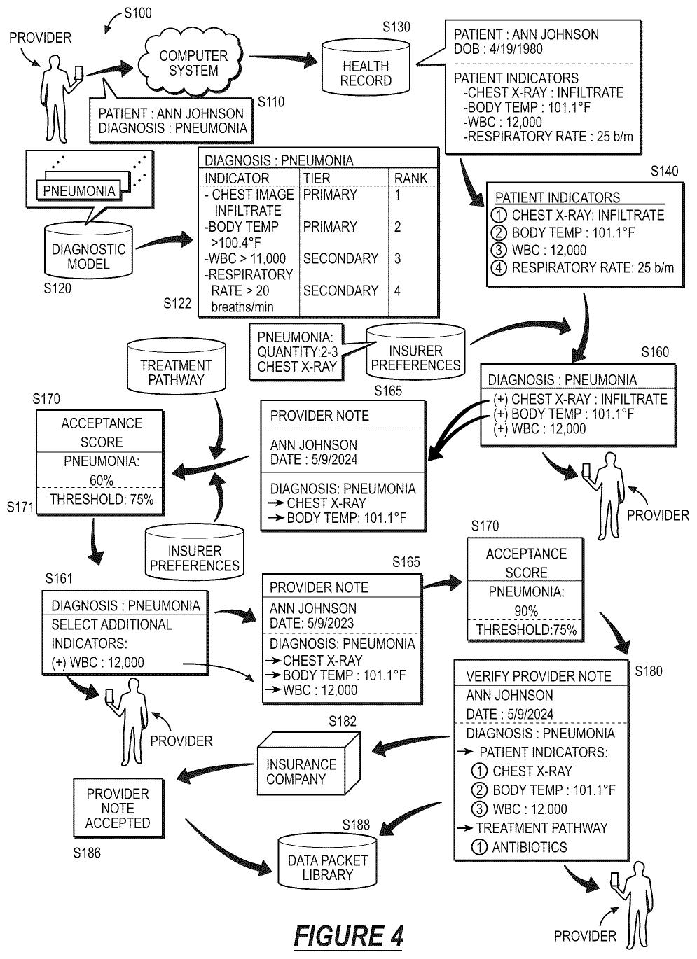

As shown in FIG. 4, one variation of the method S100 includes: receiving identification of a first diagnosis, in a population of diagnoses, for a first encounter with a first patient from an instance of a provider portal accessed by a provider in Block S110; accessing a health record, in a population of health records, corresponding to the patient and including a corpus of patient data associated with the patient in Block S130; accessing a diagnostic model including a population of modules, corresponding to a population of diagnoses, each module, in the population of modules, defining a set of target indicators supporting a corresponding diagnosis in the population of diagnoses; for a first module, in the population of modules, corresponding to the first diagnosis, accessing a first set of target indicators supporting the first diagnosis, the first set of target indicators including a set of primary target indicators supporting the first diagnosis and a set of secondary target indicators supporting the first diagnosis in combination with the set of primary target indicators in Block S122; extracting a set of primary patient indicators, from the corpus of patient data, corresponding to the set of primary target indicators and supporting the first diagnosis for the patient in Block S140; in response to a first quantity of primary patient indicators in the set of primary patient indicators exceeding a threshold quantity, populating a first notification with the set of primary patient indicators and a first prompt to select indicators, in the set of primary patient indicators, for specifying in a provider note generated for the encounter and indicating the first diagnosis for the patient, and, transmitting the first notification to the provider via the provider portal in Block S160; and, in response to receiving selection of a first subset of primary patient indicators, from the subset of primary patient indicators, in the set of primary patient indicators, appending the provider note with the subset of primary patient indicators linked to the first diagnosis in Block S165.

As shown in FIG. 4, one variation of the method S100 includes: receiving identification of a diagnosis, in a population of diagnoses, for an encounter with a patient from an instance of a provider portal accessed by a provider in Block S110; accessing a health record, in a population of health records, corresponding to the patient and including a corpus of patient data associated with the patient in Block S130; accessing a diagnostic model including a population of modules, corresponding to a population of diagnoses, each module, in the population of modules, defining a set of target indicators supporting a corresponding diagnosis in the population of diagnoses; and extracting a set of patient indicators, from the corpus of patient data, corresponding to the set of target indicators and supporting the diagnosis in Block S122. In this variation, the method S100 further includes: extracting a set of patient indicators, from the corpus of patient data, corresponding to the set of target indicators and supporting the diagnosis for the patient in Block S140; selecting a first subset of patient indicators, in the set of patient indicators, for presenting to the provider in support of the diagnosis, the first subset of patient indicators predicted to yield a threshold probability of acceptance of a provider note specifying the diagnosis and the first subset of patient indicators; generating a notification including the subset of target indicators and a prompt to review each patient indicator, in the subset of patient indicators, for specifying in the provider note generated for the encounter and indicating the diagnosis for the patient; transmitting the notification to the provider via the provider portal in Block S160; and, in response to receiving selection of a second subset of patient indicators, in the first subset of patient indicators, appending the provider note with the second subset of patient indicators linked to the diagnosis in Block S165.

As shown in FIG. 1, one variation of the method S100 includes: receiving identification of a first diagnosis, in a population of diagnoses, for a first encounter with a first patient from an instance of a provider portal accessed by a provider in Block S110; accessing a diagnostic model containing a population of modules, each module, in the population of modules, defining a particular diagnosis in the population of diagnoses in Block S120; retrieving a first module, in the population of modules, defining the first diagnosis, a first set of target indicators supporting the first diagnosis in Block S122; retrieving a timeseries of health data captured stored in an electronic health record affiliated with the patient in Block S130; and, based on the first set of target indicators, extracting a first set of indicators—configured to support the first diagnosis—from the timeseries of health data in Block S140. The method S100 further includes: based on a first set of module parameters (e.g., a minimum quantity of indicators, a required indicator type, a required combination of indicators) defined for the first module, selecting a first subset of indicators, in the first set of indicators, for presenting to the provider in support of the first diagnosis, the first subset of indicators predicted to yield a threshold probability of acceptance of a provider note (e.g., a clinical note, a progress note) specifying the first diagnosis and the first subset of indicators; and presenting the first subset of indicators—paired with the first diagnosis supplied by the provider—to the provider within the instance of the provider portal in Block S160. In one variation, the method S100 further includes, in response to the provider selecting a first indicator, in the first subset of indicators, within the instance of the provider portal, appending a first provider note—generated for the encounter and specifying the first diagnosis—with the first indicator.

### 1.1 Method: Predicting a New Diagnosis

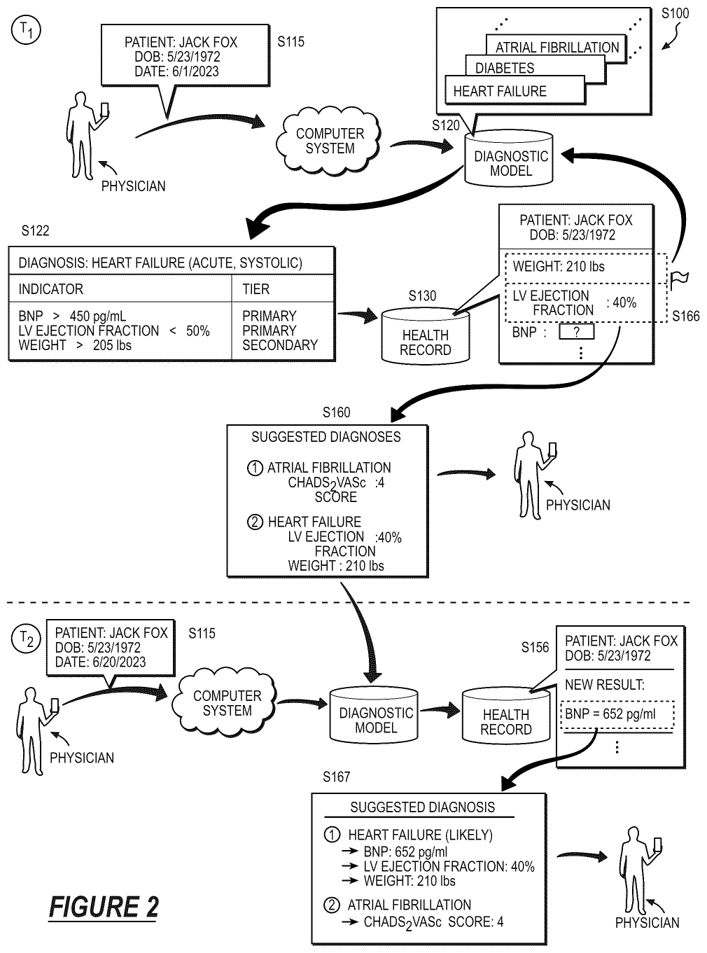

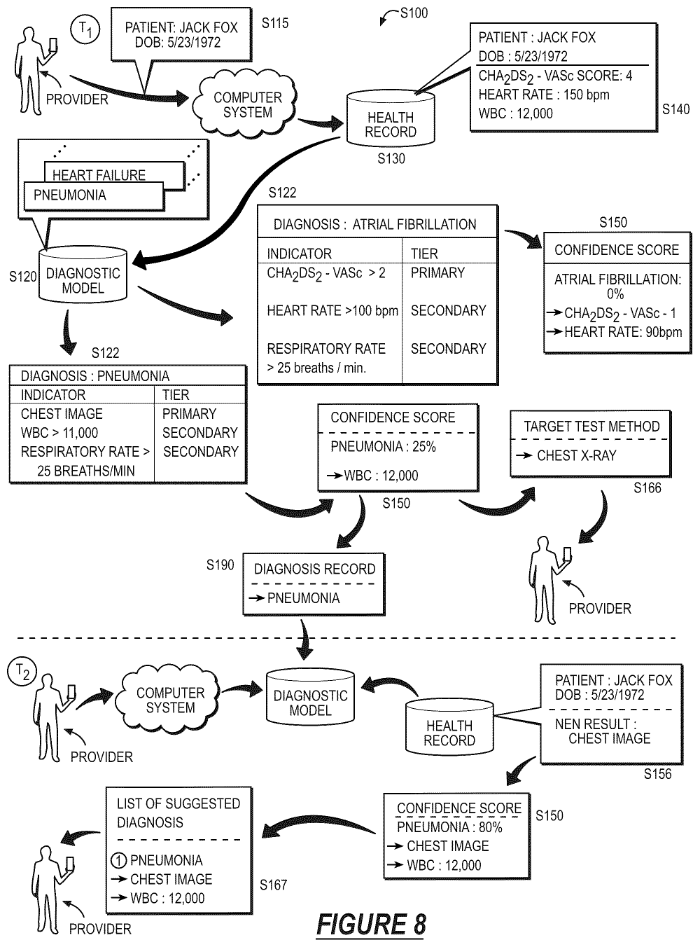

As shown in FIGS. 2, 3, and 7-10, one variation of the method S100 includes, in response to receiving confirmation of a patient encounter from a provider via an instance of the provider portal: accessing a health record, in a population of health records, corresponding to the patient, the health record including a corpus of patient data associated with the patient in Block S130; accessing a diagnostic model including a population of modules, corresponding to a population of diagnoses, each module, in the population of modules, defining a set of target indicators supporting a corresponding diagnosis in the population of diagnoses in Block S120; for a first module, in the population of modules, corresponding to a first diagnosis, accessing a first set of target indicators supporting the first diagnosis in Block S122; extracting a first subset of patient indicators, from the corpus of patient data, corresponding to a first subset of target indicators in the first set of target indicators in Block S140; and deriving a first confidence score for the first diagnosis for the patient based on the first subset of patient indicators and the first subset of target indicators in Block S150. In this variation, the method S100 further includes, in response to the first confidence score exceeding a threshold score: appending a list of predicted diagnoses with the first diagnosis; generating a first notification including the list of predicted diagnoses and a first prompt to review the list of predicted diagnoses; populating the first notification with the first subset of patient indicators linked to the first diagnosis; and, via the provider portal, transmitting the first notification to the provider for review in Block S167.

In one variation, the method S100 further includes, in response to selection of the first diagnosis by the provider, generating a provider note—specifying the first diagnosis—for the patient encounter.

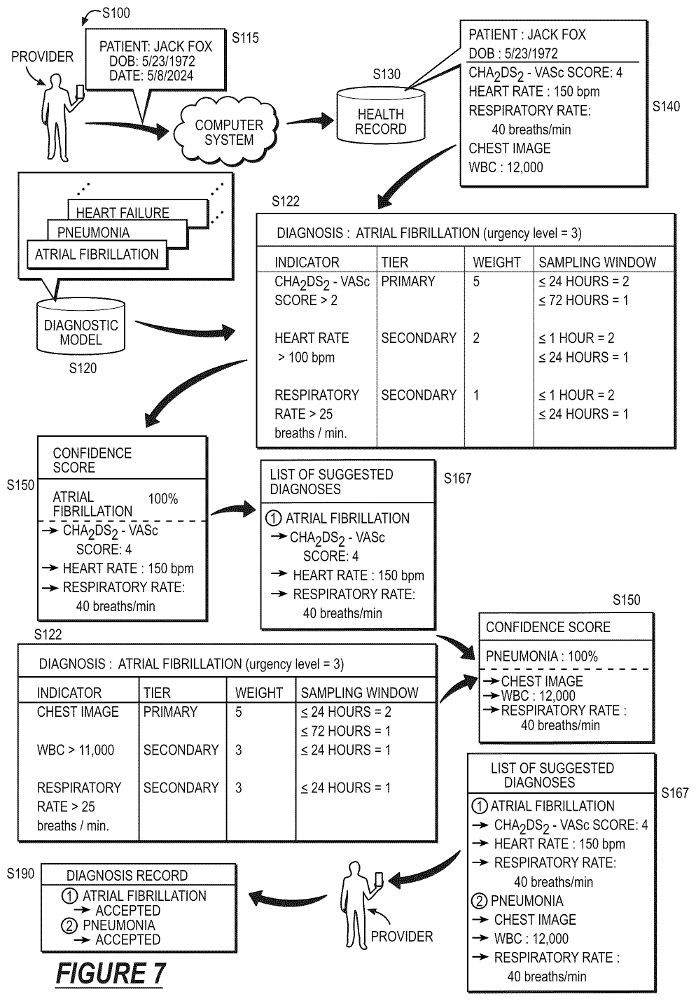

As shown in FIG. 7, one variation of the method S100 includes, in response to receiving confirmation of a patient encounter from a provider via an instance of the provider portal: accessing a health record, in a population of health records, corresponding to the patient, the health record including a corpus of patient data associated with the patient in Block S130; and accessing a diagnostic model including a population of modules, corresponding to a population of diagnoses, each module, in the population of modules defining a set of target indicators supporting a corresponding diagnosis in the population of diagnoses in Block S120. In this variation, the method S100 further includes, for a module, in the population of modules, corresponding to a diagnoses, accessing a set of target indicators including: a subset of primary target indicators supporting the diagnosis and required for predicting the diagnosis; a subset of secondary target indicators supporting the diagnosis in Block S122; and extracting a subset of primary patient indicators, from the corpus of patient data, corresponding to the subset of primary target indicators in Block S140. In this variation, the method S100 further includes, in response to the subset of primary patient indicators corresponding to the subset of primary target indicators: extracting a subset of secondary patient indicators, from the corpus of patient data, corresponding to the subset of secondary target indicators; and deriving a confidence score for the diagnosis for the patient based on the subset of primary patient indicators, the subset of secondary patient indicators, the subset of primary target indicators, and the subset of secondary target indicators in Block S150. In this variation, the method S100 further includes, in response to the confidence score exceeding a threshold score: appending a list of predicted diagnoses with the diagnosis; generating a notification including the list of predicted diagnoses and a prompt to review the list of predicted diagnoses; populating the notification with the subset of primary patient indicators and the subset of secondary patient indicators linked to the diagnosis; and, via the provider portal, transmitting the notification to the provider for review in Block S167.

As shown in FIG. 2, one variation of the method S100 includes, in response to receiving confirmation of a patient encounter from a provider via an instance of the provider portal: retrieving a diagnostic model containing a population of modules, each module, in the population of modules, defining a particular diagnosis in a population of diagnoses in Block S120; for a first module, in the population of modules, defining a first diagnosis and a set of target indicators corresponding to the first diagnosis, extracting a first set of primary target indicators, in the set of target indicators, required for supporting the first diagnosis in Block S122; accessing an electronic health record—including a timeseries of health data—affiliated with a patient in Block S130; in response to the timeseries of health data defining a first set of indicators corresponding to the set of target indicators, accessing a set of secondary target indicators, in the set of target indicators, supporting the first diagnosis; and, in response to the timeseries of health data defining a second set of indicators corresponding to a subset of secondary target indicators, in the set of secondary target indicators, appending a list of possible diagnoses with the first diagnosis and presenting the list of possible diagnoses to the provider via the instance of the provider portal for review in Block S167. In one variation, the method S100 further includes, in response to absence of indicators in the timeseries of health data corresponding to the set of primary target indicators, rejecting the first diagnosis for further investigation.

### 1.2 Method: New Diagnosis+Medication Check

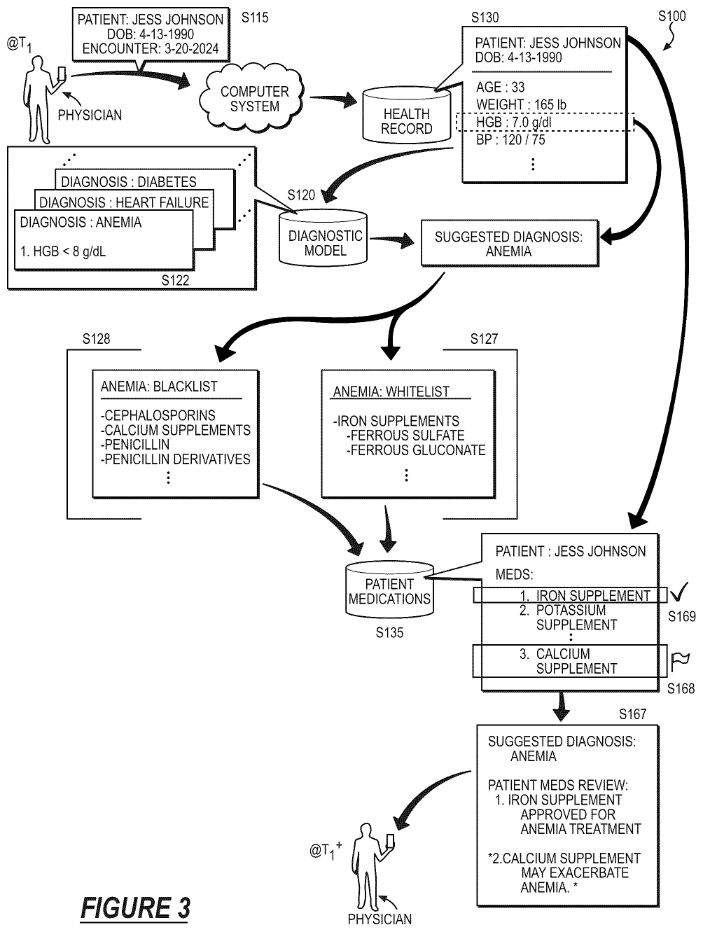

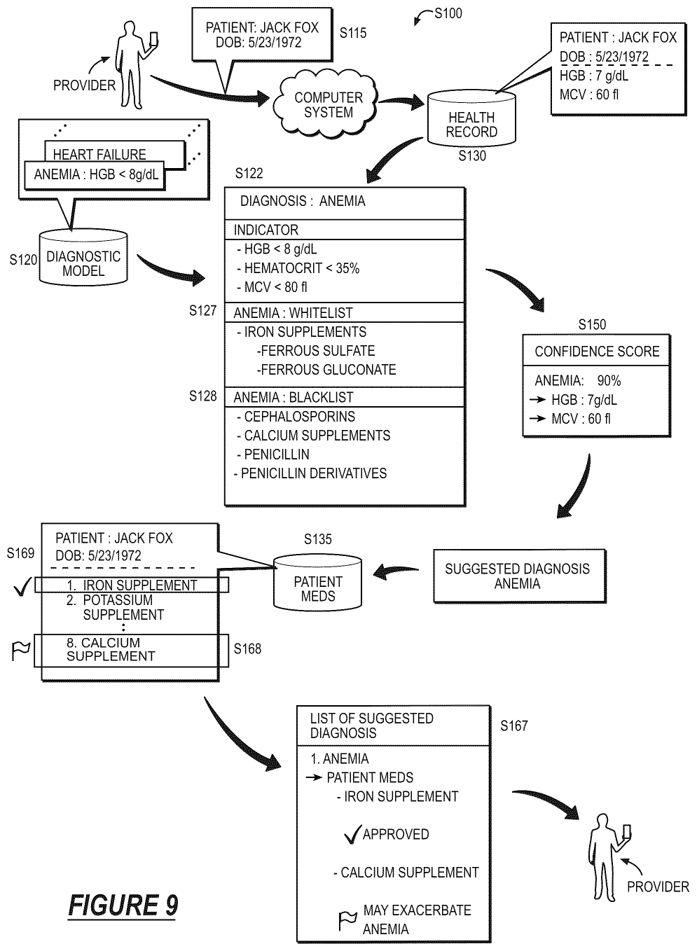

As shown in FIGS. 3 and 9, one variation of the method S100 includes, in response to receiving confirmation of a patient encounter from a provider via an instance of the provider portal: accessing a health record, in a population of health records, corresponding to the patient, the health record including a corpus of patient data and a list of current medications implemented by the patient in Block S130; and accessing a diagnostic model including a population of modules, corresponding to a population of diagnoses, each module, in the population of modules defining a set of target indicators supporting a corresponding diagnosis in the population of diagnoses in Block S120. In this variation, the method S100 further includes, for a first module, in the population of modules, corresponding to a first diagnosis: accessing a first set of target indicators supporting the first diagnosis, and a medication blacklist including a set of blacklisted medications predicted to exacerbate the first diagnosis in Block S122; extracting a first subset of patient indicators, from the corpus of patient data, corresponding to a first subset of target indicators in the first set of target indicators in Block S140; and deriving a first confidence score for the first diagnosis for the patient based on the first subset of patient indicators and the first subset of target indicators in Block S150. In this variation, the method S100 further includes, in response to the first confidence score exceeding a threshold score: predicting the first diagnosis for the patient for the encounter; in response to predicting the first diagnosis for the patient, appending a list of predicted diagnoses with the first diagnosis, and generating a first notification including the list of predicted diagnoses and a first prompt to review the list of predicted diagnoses. In this variation, the method S100 further includes, in response to the list of current medications including a first medication, in the set of blacklisted medications: flagging the first medication for review by the provider; and populating the first notification with a first alert indicating implementation of the first medication by the patient; and, via the provider portal, transmitting the first notification to the provider for review in Block S167.

### 2. Applications: Live Diagnosis+Provider Note Augmentation

Generally, Blocks of the method S100 can be executed by a computer system (e.g., a remote computer system, a computer network, a remote server) in conjunction with a health care platform (e.g., an electronic health record platform) and/or application (e.g., native or web application) to: receive a diagnosis from a provider during and/or immediately succeeding an encounter with a patient; scan the patient's health record for evidence—such as including demographic data (e.g., age, sex, geographic location), test data (e.g., historical and/or current vitals, lab results, imaging results), lifestyle data (e.g., stress level, activity level), medication data, etc.—supporting the diagnosis supplied by the provider; and, in (near) real-time, selectively present a list of evidence supporting the diagnosis—and extracted from the patient's health record—to the provider for review and/or selection for specifying in a provider note (e.g., a progress note, a clinical note, an admission note, a discharge summary) generated for the encounter with the patient. Furthermore, in response to receiving selection of a subset of evidence in the list of evidence presented to the provider, the computer system can automatically append the provider note with this subset of evidence.

In particular, a provider may specify a particular diagnosis for the patient during the encounter (e.g., a healthcare encounter), such as via a provider portal executing on a computing device (e.g., a tablet, a desktop computer, a smartphone) accessed by the provider. The computer system can interface with the provider portal to receive the diagnosis and—in (near) real-time—derive a list of evidence and/or combinations of evidence from the patient's health record that support the diagnosis. The computer system can then selectively present this list of evidence to the provider (e.g., within the provider portal) in order to: promote review of evidence and/or selection of evidence by the provider for pairing with the diagnosis in a provider note generated for the encounter, such as by selectively surfacing key evidence and limiting presentation of nonessential evidence to the provider, which may result in “note bloat”; reduce resources (e.g., time) dedicated by the provider generating the provider note and therefore increase resources available for the provider to dedicate to patient care; and promote pairing of the diagnosis with specific types and/or combinations of evidence—selected by the provider from the list of evidence presented within the provider portal—in the provider note in order to meet a threshold criteria associated with acceptance of provider notes (e.g., by an insurance company employed by the patient) specifying the diagnosis.

In one implementation, the computer system can implement a diagnostic model loaded with a population of modules—representative of a population of diagnoses—each module, in the population of modules, defining: a particular diagnosis in the population of diagnoses; a set of target indicators (i.e., units and/or combinations of evidence)—such as derived from timeseries health data including vital data (e.g., body temperature, pulse rate, respiration rate, blood pressure, weight), lab data (e.g., white blood cell count, culture presence), image data (e.g., X-ray data, CT scan data), demographic information (e.g., age, sex, geographic location), lifestyle data (e.g., activity level, sleep level, stress level), medication data, etc.—that support the particular diagnosis; and a set of treatment pathways (e.g., medications, procedures, lifestyle changes, activities) associated with treatment of the diagnosis.

In this implementation, in response to receiving identification of a diagnosis from the provider via the provider portal, the computer system can therefore: automatically retrieve a corresponding module, in the population of modules, defining the diagnosis specified by the provider; and identify a set of target indicators—defined by the corresponding module—supporting the diagnosis. The computer system can then: retrieve a health record (e.g., an electronic health record) of a patient affiliated with the diagnosis; and scan the health record for indicators (e.g., vitals, lab results, image results, lifestyle factors, demographics, medications)—derived from timeseries health data contained in the health record—corresponding to the set of target indicators defined for the diagnosis. The computer system can thus: extract a set of indicators—corresponding to one or more target indicators in the set of target indicators—from the health record; and selectively present the set of indicators to the provider—for pairing with the diagnosis—within the provider portal.

For example, the computer system can selectively present a subset (e.g., one, two, ten) of indicators, in the set of indicators, to the provider for pairing with the diagnosis in order to promote selection of: a threshold quantity of indicators required to support the first diagnosis in a provider note; a particular combination of indicators, in the set of indicators, required to support the first diagnosis in a provider note; a first indicator, in the set of indicators, required to support the first diagnosis and a subset of indicators, in the set of indicators, required to achieve the threshold quantity of indicators in combination with the first indicator; etc. In particular, in this example, the computer system can therefore: prioritize surfacing of key indicators that support the diagnosis and therefore aid the provider in generating a comprehensive provider note with sufficient context for the provider and/or other providers to reference, thereby improving and/or increasing efficiency of patient care; minimize an amount of “nonessential” indicators surfaced to the provider and thereby prioritize review of only key or “essential” indicators by the provider; and promote appending of the provider note with a particular indicator and/or combination of indicators—selected by the provider from the subset of indicators—configured to support the diagnosis and predicted to yield acceptance (e.g., by an insurance company) of the provider note. The computer system can then receive selection of one or more indicators—in the subset of indicators presented to the provider—from the provider via the provider portal; and append a provider note—generated for the encounter with the patient—with the selected indicators accordingly.

Additionally, in one implementation, the computer system can predict an acceptance score—representing a likelihood of acceptance of the first diagnosis as characterized in the provider note, such as by an insurance company affiliated with the patient—for the provider note based on a particular subset of indicators selected by the provider for specifying in the provider note. For example, the computer system can: present a set of patient indicators—extracted from the patient's health record—to the provider (e.g., via the provider portal) in support of a diagnosis supplied by the provider; receive selection of a subset of patient indicators, in the set of patient indicators, for including in a provider note generated for the encounter and indicating the diagnosis; and, based on the subset of patient indicators selected by the provider, predict an acceptance score—representing a likelihood of acceptance of the diagnosis for the patient and/or acceptance of the provider note (e.g., by the patient's insurance company)—for the provider note. The computer system can then selectively: verify the provider note in response to the acceptance score exceeding a threshold score; or withhold verification of the provider note—in response to the acceptance score falling below the threshold score—and/or prompt the provider to select and/or obtain additional patient indicators supporting the diagnosis for including the provider note. The computer system can therefore: increase the likelihood of acceptance of the diagnosis and/or provider note acceptance (e.g., by an insurance company affiliated with a patient); minimize resources dedicated to generating provider notes (i.e., medical documentation); and minimize resources dedicated to amending rejected provider notes (i.e., rejected by insurance company).

### 2.1 Applications: New Diagnosis

In one variation, Blocks of the method S100 can be executed by a computer system in conjunction with a health care platform and/or application to: receive identification of a patient from a provider via a provider portal; retrieve a health record (e.g., an electronic health record)—including demographic data (e.g., age, sex, geographic location), timestamped test data (e.g., historical and/or current vitals, lab results, imaging results), lifestyle data (e.g., stress level, activity level), medication data, etc.—corresponding to the patient; access a diagnostic model including a population of modules—corresponding to a population of diagnoses—defining target indicators (i.e., evidence) and/or combinations of target indicators supporting each diagnosis in the population of diagnoses; scan the patient's health record for indicators corresponding to (e.g., matching, falling within a particular range, meeting a threshold criteria) these target indicators; and predict a particular diagnosis (or diagnoses) for the patient based on correlations between indicators (e.g., a blood pressure reading, a resting heart rate, a white blood cell count, a body temperature, an oxygen saturation) derived from the patient's health record and target indicators defined by a module, in the population of modules, corresponding to the particular diagnosis. The computer system can then: append a list of predicted diagnoses with the particular diagnosis; and serve this list of predicted diagnoses to the provider for review and/or approval or rejection.

In one implementation, the computer system can implement a diagnostic model loaded with a population of modules—representative of a population of diagnoses—each module, in the population of modules, defining: a particular diagnosis in the population of diagnoses; a set of target indicators (i.e., units and/or combinations of evidence)—such as derived from timeseries health data including vital data (e.g., body temperature, pulse rate, respiration rate, blood pressure, weight), lab data (e.g., white blood cell count, culture presence), image data (e.g., X-ray data, CT scan data), demographic information (e.g., age, sex, geographic location), lifestyle data (e.g., activity level, sleep level, stress level), medication data, etc.—that support the particular diagnosis; and a set of treatment pathways (e.g., medications, procedures, lifestyle changes, activities) associated with treatment of the diagnosis.

For example, in response to receiving identification of the patient from the provider—such as before, during, or after an encounter with the patient—the computer system can: scan all contents of the patient's health record, such as including all recent and historical health data collected for this patient over time; represent data retrieved from the patient's health record as a population of indicators (e.g., according to a standardized format); retrieve a first module, in the population of modules, corresponding to a first diagnosis, in the population of diagnoses, and defining a first set of target indicators supporting the first diagnosis; search for the first set of target indicators—such as in a particular order or grouping defined by a set of module parameters defined for the first module—in the set of indicators; and selectively flag and/or withhold flagging of the first diagnosis as a predicted diagnosis based on identification of the first set of target indicators in the set of indicators derived from the patient's health record. The computer system can then: repeat this process for each other module, in the population of modules, to flag and/or withhold flagging of each diagnosis, in the population of diagnoses, as a possible diagnosis and therefore generate a robust list possible diagnoses for this patient; and present this list of possible diagnoses—such as in a particular order based on urgency, confidence in diagnosis, relevancy to a current encounter, etc.—for review within the provider portal in (near) real-time.

### 2. Provider Portal+Provider Notes

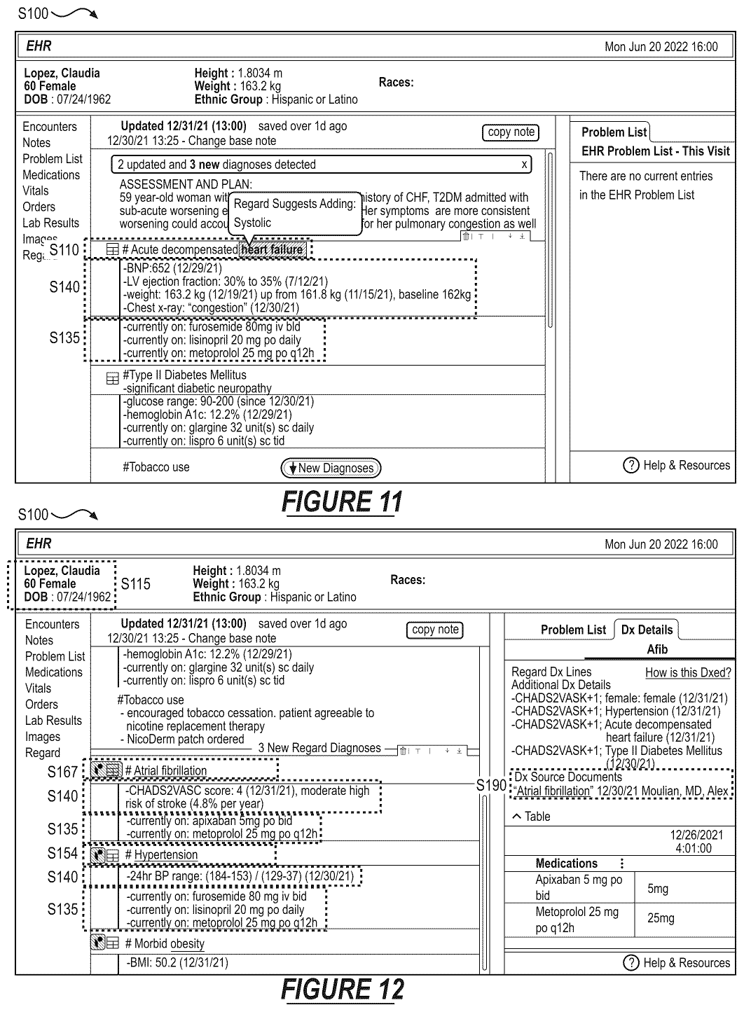

Generally, as shown in FIGS. 11 and 12, the computer system can host or interface with a provider portal (e.g., a native application or web application) executing on a computing device (e.g., a mobile device, a computer) accessed by a provider to selectively guide and/or support the provider in generating a provider note (hereinafter a “note”) for a patient encounter.

In one implementation, the computer system can integrate with a cloud platform employed by a health care system (e.g., a hospital or hospital system, a clinic, a health care group)—such as an electronic health record (or “EHR”) platform—in order to access health records (e.g., EHRs) containing historical and/or current health information of patients affiliated with the health care system. For example, the computer system can integrate with an EHR platform employed by the health care system and therefore access a population of health records corresponding to a population of patients affiliated with the health care system, each health record, in the population of health records, containing timeseries patient data captured for a patient, in the population of patients, over time and including: a list of timestamped encounters for this patient at a facility affiliated with the health care system; a series of timestamped notes—each note corresponding to a timestamped encounter in the list of timestamped encounters—previously generated for this patient; a list of timestamped medications prescribed and/or employed by the patient (e.g., historically and/or currently); a series of timestamped vital data (e.g., heart rate, respiratory rate, body temperature, oxygen saturation) recorded for the patient; a series of timestamped test results—including labs and/or images—obtained for the patient; a list of timestamped medical diagnoses or symptoms exhibited by the patient; etc.

Generally, the computer system can host or interface with the provider portal—accessed by the provider—to aid the provider in producing a note (e.g., an electronic progress note, an electronic clinical note) containing relevant information for a corresponding encounter (or “patient encounter”) between a patient and the provider. In particular, the computer system can interface with the provider portal to: compile a set of note data—including a diagnosis, a set of evidence (or “indicators”) supporting the diagnosis, and/or a treatment pathway—provided and/or selected by the provider within the provider portal; and append a note—generated for a particular encounter between the provider and a patient—with the set of note data accordingly. In one example, upon generation of the note, the computer system can prompt the provider to verify (e.g., review and sign) the note and then finalize the note—such as for transmitting to a health insurance agency associated with the patient for review and/or for storage in a note database—responsive to receiving verification from the provider accordingly.

### 4. Diagnostic Model

Generally, the computer system can implement a diagnostic model linking each diagnosis, in a population of diagnoses, to a set of supporting evidence (or “indicators”) that support the diagnosis and/or to a set of treatment pathways corresponding to the diagnosis. For example, the computer system can implement a diagnostic model linking: a first diagnosis to a first set of target indicators—such as derived from patient vitals, labs, images, etc.—and a first set of treatment pathways (e.g., medications, procedures, lifestyle changes) associated with the first diagnosis; a second diagnosis to a second set of indicators and a second set of treatment pathways associated with the second diagnosis; a third diagnosis to a third set of indicators and a third set of treatment pathways associated with the third diagnosis; etc. The computer system can therefore implement a diagnostic model that links each diagnosis, in the population of diagnoses, to target indicators (i.e., evidence) and/or combinations of target indicators that support the diagnosis and treatment pathways configured to treat and/or mitigate the diagnosis, and thereby represents key information (e.g., diagnoses, target indicators, treatment pathways) required for generation and/or acceptance of notes.

In one implementation, the diagnostic model can include a population of modules, each module, in the population of modules, corresponding to a particular diagnosis in a population of diagnoses. In particular, each module (e.g., a data container), in the population of modules, can define: a diagnosis; a set of target indicators associated with (e.g., supporting) the diagnosis; and a set of treatment pathways associated with the diagnosis.

For example, the diagnostic model can include a population of modules including a first module defining: a first diagnosis of pneumonia; a first set of target indicators—such as including a target body temperature, a target white blood cell count, a target oxygen saturation, a target chest image (e.g., a target chest x-ray, a target chest CT), a target procalcitonin level, etc.—associated with pneumonia; and a first set of treatment pathways—such as including a set of antibiotics and/or a set of medical procedures (e.g., intubation, extubation)—associated with treatment and/or mitigation of pneumonia. The population of modules can further include a second module defining: a second diagnosis of acute, systolic, congestive heart failure; a second set of target indicators—such as including a target left ventricular ejection fraction (or “LVEF”), a target body weight, a target B-type natriuretic peptide (or “BNP”) level, a target chest x-ray, etc.—associated with acute, systolic, congestive heart failure; and a second set of treatment pathways—such as including a set of medications—associated with treatment and/or mitigation of acute, systolic, congestive heart failure. Furthermore, the population of modules can include a third module defining: a third diagnosis of Type II Diabetes Mellitus (or “adult onset diabetes”); a third set of target indicators—such as including a target glucose level, a target hemoglobin A1C (or “HbA1c”) level, a target diabetic neuropathy level (e.g., specified by the patient)—associated with Type II Diabetes Mellitus; and a third set of treatment pathways—such as including a set of insulin medications—associated with treatment and/or mitigation of Type II Diabetes Mellitus.

The computer system can thus store the first, second, and third modules within the diagnostic model. Furthermore, the computer system can store additional modules (e.g., hundreds, thousands, tens of the thousands)—corresponding to an expansive population of diagnoses—within the diagnostic model, such that the diagnostic model defines a comprehensive library of diagnosis modules, each diagnosis module defining a medical diagnosis, corresponding indicators associated with the diagnosis, and a corresponding treatment pathway(s) configured to mitigate the diagnosis.

### 4.1 Supporting Evidence: Target Indicators

Generally, the computer system can implement a diagnostic model linking each diagnosis, in a population of diagnoses (e.g., medical diagnoses), to units and/or combinations of evidence (e.g., vitals, lab results, imaging results) that support the diagnosis. In particular, the computer system can implement a diagnostic model storing a population of modules, each module, in the population of modules, defining a particular diagnosis, in the population of diagnoses, and a set of target indicators (i.e., supporting evidence) corresponding to (e.g., supporting) the particular diagnosis.

For example, the computer system can implement a diagnostic model including a first module, in a population of modules, defining a first diagnosis and a first set of target indicators corresponding to the first diagnosis, such as including: a set of target vital signs (e.g., body temperature, pulse rate, respiration rate, blood pressure, weight); a set of target lab results (e.g., white blood cell count, culture presence); a set of target imaging results (e.g., X-ray result, CT result); a set of target lifestyle factors (e.g., smoker, nonsmoker, activity level, stress level); a set of target demographics (e.g., age, sex, geographic location); and/or a set of target medications currently prescribed and/or employed by a patient. The computer system can therefore retrieve this first module—stored in the diagnostic model—to identify key indicators and/or types of indicators that support the first diagnosis. Furthermore, in this example, the population of modules can further include: a second module defining a second diagnosis and a second set of target indicators corresponding to the second diagnosis; a third module defining a third diagnosis and a third set of target indicators corresponding to the third diagnosis; etc.

In one implementation, the computer system can implement a diagnostic model linking each diagnosis, in the population of diagnoses, to distinct combinations of evidence that support the diagnosis. In particular, in this implementation, the computer system can access a diagnostic model including a first module, in the population of modules, defining a first diagnosis and one or more combinations of evidence (i.e., target indicators) that support the first diagnosis. For example, the first module can define: a first combination of evidence—including a first subset of target indicators—that, in combination, support the first diagnosis; a second combination of evidence—including a second subset of target indicators—that, in combination, support the first diagnosis; and a third combination of evidence—including a third subset of target indicators—that, in combination, support the first diagnosis. In this implementation, each other module, in the population of modules, can similarly define a diagnosis and one or more combinations of target indicators that cooperate to support the diagnosis.

### 4.1.1 Dynamic Evidence: Patient Characteristics

In one variation, the computer system can implement a diagnostic model including a first module, in a population of modules, defining a first diagnosis and one or more combinations of evidence that support the first diagnosis based on a set of patient characteristics, such as including patient demographic data (e.g., age, sex, geographic location), lifestyle data (e.g., activity level, stress level, average amount of sleep), historical health data (e.g., medications, historical diagnoses, smoker or non-smoker), etc.

For example, the first module can define a first set of target indicators—such as a target resting heart rate within a first range and/or a target blood pressure within a first range—for a particular diagnosis and for a patient exhibiting a first set of target patient characteristics including: an age within a first age range (e.g., between ages 18 and 35); a first sex (e.g., male); and status as a non-smoker. In this example, the first module can further define: a second set of target indicators—such as a target resting heart rate within a second range and/or a target blood pressure within a second range—for the particular diagnosis and for a patient exhibiting a second set of target patient characteristics including an age within a second age range (e.g., between ages 35 and 45), the first sex (e.g., male), and status as a non-smoker; a third set of target indicators—such as a target resting heart rate within a third range and/or a target blood pressure within a third range—for the particular diagnosis and for a patient exhibiting a third set of target patient characteristics including an age within the first age range (e.g., between ages 35 and 45), a second sex (e.g., female), and status as a non-smoker; etc. In this example, the first module can therefore define a different set of target indicators for different types and/or combinations of patient characteristics, such as whether the patient is within a first or second age group, whether the patient is male or female, and whether the patient is a smoker or a non-smoker.

### 4.1.2 Tiered Evidence

Additionally or alternatively, in another implementation, the computer system can implement a diagnostic model defining a set of tiered evidence for each diagnosis in the population of diagnoses. In particular, in this implementation, the computer system can implement a diagnostic model including a first module, in a population of modules, defining: a first diagnosis; a set of target indicators supporting the first diagnosis and organized within a set of tiers—such as including a primary tier, a secondary tier, and/or a tertiary tier—each successive tier, in the set of tiers, configured to be combined with each preceding tier. For example, the first module can define the set of target indicators including: a primary target indicator supporting the first diagnosis; and a secondary target indicator supporting the first diagnosis when combined with the primary target indicator. Therefore, in this example, the first module can define: the primary target indicators as supporting the first diagnosis independent of the secondary target indicator; and the secondary target indicator as supporting the first diagnosis dependent on presence of the primary target indicator.

In one example, the computer system can implement a diagnostic model including a pneumonia module defining: a pneumonia diagnosis; a primary target indicator corresponding to a positive chest image including positive indication of infiltrate, consolidation, density, or opacity within a patient's chest; and a secondary target indicator corresponding to a body temperature exceeding a threshold temperature (e.g., 100.4 degrees fahrenheit). In this example, the computer system can therefore selectively consider and/or disregard body temperature data—specified for a patient in the patient's EHR—based on whether the patient's EHR includes health data (e.g., images, image results) that indicates recordation of a positive chest image for the patient (e.g., within a target sampling period).

### 4.1.2 Target Sampling Window

Additionally or alternatively, in another implementation, the computer system can implement a diagnostic model defining a target sampling window—such as between a time of recordation of a particular health indicator and a time of diagnosis—for each target indicator and/or for a set of target indicators defined for the diagnosis. In particular, in one example, a first module can define: a first diagnosis; a first target indicator defining a 24-hour sampling window, such that the first target indicator only supports the first diagnosis if obtained within a 24-hour time period preceding a current patient encounter; and a second target indicator defining a one-year sampling window, such that the second target indicator only supports the first diagnosis if obtained within a 1-year time period preceding the current patient encounter.

Additionally or alternatively, in another implementation, the computer system can implement a diagnostic model assigning a particular weight to each target indicator based on an age of health data—contained in the patient's health record—corresponding to the target indicator. For example, the diagnostic model can include a first module defining: a first diagnosis; a first target indicator defining a first weight when captured within a first target sampling window (e.g., 1 hour, 24 hours) and a second weight, less than the first weight, when captured within a second target sampling window (e.g., 3 days, 1 week, 1 month, 1 year) exceeding the first target sampling window. The computer system can therefore implement the diagnostic model to: assign the first weight to a first indicator corresponding to the first target indicator and captured within the first target sampling window specified by the first module; and assign the second weight to a second indicator corresponding to the first target indicator and captured within the second target sampling window specified by the first module.

Therefore, in the preceding implementations, the computer system can implement the diagnostic model to selectively consider or disregard health data—corresponding to target indicators defined by the diagnostic model—contained in a patient's health record based on a duration between collection of relevant health data and diagnosis.

### 4.1.4 Diagnosis Risk

Additionally, in one variation, the computer system can implement a diagnostic model defining risk (e.g., likelihood, severity) of a particular diagnosis based on detection of different combinations of target indicators associated with the particular diagnosis within the diagnostic model. In particular, in this variation, each module, in the population of modules contained within the diagnostic model, can define risk—such as a “negative” diagnosis, a “resolved” diagnosis, a “possible” diagnosis, a “probable” diagnosis, or a “positive” diagnosis—of a corresponding diagnosis based on presence of a particular subset of target indicators in a set of target indicators supporting the corresponding diagnosis.

In one example, the diagnostic model can include a first module, in a population of modules, defining: a first diagnosis; a primary target indicator that supports the first diagnosis; a first secondary target indicator and a second secondary target indicator that support the first diagnosis when combined with the primary target indicator. In this example, the first module can define: detection of the primary target indicator as corresponding to a “possible” diagnosis; detection of the primary target indicator and the first secondary target indicator as corresponding to a “probable” diagnosis; detection of the primary target indicator, the first secondary target indicator, and the second secondary target indicator as corresponding to a “positive” diagnosis; and detection of the first and/or second secondary target indicator—omitting the primary target indicator—as corresponding to a “negative” diagnosis.

### 4.1.5 Module+Submodules: Diagnosis+Types of Diagnosis

Additionally or alternatively, in one variation, the computer system can implement a diagnostic model including a population of modules and a population of submodules—corresponding to specific types and/or subtypes of generic diagnoses represented in the population of modules—linked to and/or integrated within the population of modules. In particular, in this implementation, the computer system can implement a diagnostic model including a population of modules, each module in the population of modules: defining a primary diagnosis (e.g., a generic and/or broad diagnosis); and including a set of submodules linked to and/or integrated within the module, each submodule, in the set of submodules, defining a secondary diagnosis, corresponding to a particular type of the primary diagnosis, and a set of target indicators supporting the secondary (and primary) diagnosis. The diagnostic model can therefore be configured to: represent a primary diagnosis—defining a first resolution (e.g., degree of specificity)—within a module in the population of modules; and represent a set of secondary diagnoses—corresponding to the primary diagnosis and defining a second resolution exceeding the first resolution—within a set of submodules linked to the module.

For example, the computer system can implement the diagnostic model including: a module defining a primary diagnosis of heart failure and a first set of target indicators associated with heart failure; a first submodule—linked to the module—defining a first secondary diagnosis of systolic heart failure, a first set of target indicators associated with systolic heart failure, and/or a first set of treatment pathways configured to mitigate systolic heart failure and/or symptoms of systolic heart failure; and a second submodule—linked to the module—defining a second secondary diagnosis of diastolic heart failure, a second set of target indicators associated with diastolic heart failure, and/or a second set of treatment pathways configured to mitigate diastolic heart failure and/or symptoms of diastolic heart failure.

### 4.1.6 Example: Pneumonia Module

In one example, the computer system can implement a diagnostic model including a “pneumonia” module representative of different types and/or severities of pneumonia. In particular, the diagnostic model can include the pneumonia module defining: a primary diagnosis of pneumonia; and a first set of target indicators associated with the primary diagnosis of pneumonia. In particular, in this example, the pneumonia module can define the first set of target indicators including: a primary target indicator corresponding to a positive chest image (e.g., an x-ray, a CT) that includes positive indication of infiltrate, consolidation, density, or opacity within a patient's chest; a secondary target indicator corresponding to a body temperature exceeding 100.4 degrees fahrenheit within 48 hours of confirmation of a positive chest image (i.e., the first target indicator); and a set of tertiary target indicators including a target white blood cell count (or “WBC”) exceeding 11,000 within 24 hours of a positive chest image (i.e., the first target indicator), a target oxygen saturation (or “SpO2”) less than 88% within 24 hours of a positive chest image; and/or a target respiratory rate exceeding 20 breaths-per-minute within 24 hours of a positive chest image.

Furthermore, the pneumonia module can include a first set of submodules—linked to and/or integrated within the pneumonia module—including: a first submodule defining a first secondary diagnosis of “community-acquired” pneumonia and defining a first set of target indicators supporting the secondary diagnosis; a second submodule defining a second secondary diagnosis of “hospital-acquired” pneumonia and defining a second set of target indicators supporting the secondary diagnosis; and a third submodule defining a third secondary diagnosis of “ventilator-acquired” pneumonia and defining a third set of target indicators supporting the secondary diagnosis. The pneumonia module can further include a second set of submodules—linked to and/or integrated within the pneumonia module—including: a fourth submodule defining a first tertiary diagnosis of “viral” pneumonia and defining a fourth set of target indicators supporting the first tertiary diagnosis; a fifth submodule defining a second tertiary diagnosis of “fungal” pneumonia and defining a fifth set of target indicators supporting the second tertiary diagnosis; and a sixth submodule defining a third tertiary diagnosis of “bacterial” pneumonia and defining a sixth set of target indicators supporting the third tertiary diagnosis. For example, additional target indicators—defined in the first and second set of submodules—can include: a respiratory culture positive for bacterial organisms; a legionella urinary antigen positive; a streptococcal pneumoniae urinary antigen positive; a procalcitonin level exceeding 0.5 ug/L; a respiratory viral panel positive for viral organism; an influenza A/B/RSV swab positive; a SARS-CoV-2 swab positive; a beta-D-glucan positive; a galactomannan positive; etc.

### 4.2 Treatment Pathway

Furthermore, each module, in the population of modules can define one or more treatment pathways corresponding to a diagnosis defined by the module. In particular, each module in the population of modules can define: a particular diagnosis in the population of diagnoses; a set of target indicators supporting the particular diagnosis; and a set of treatment pathways—associated with treatment and/or mitigation of the diagnosis and/or one or more target indicators in the set of target indicators—corresponding to the diagnosis. For example, the computer system can implement a diagnostic model including a first module, in a population of modules, defining a first diagnosis, a first set of target indicators, and a first set of treatment pathways—associated with treatment and/or mitigation of the diagnosis—including: a first treatment pathway associated with mitigation of the diagnosis; a second treatment pathway associated with mitigation of the diagnosis; a third treatment pathway associated with alleviation and/or mitigation of patient symptoms associated with a first subset of target indicators in the set of target indicators; and/or a fourth treatment pathway associated with alleviation and/or mitigation of patient symptoms associated with a second subset of target indicators in the set of target indicators.

Additionally or alternatively, in one implementation, the computer system can implement a diagnostic module defining one or more treatment pathways for distinct combinations of evidence (i.e., target indicators) supporting a particular diagnosis. For example, in this implementation, the computer system can implement a diagnostic module including a first module, in a population of modules, defining: a first diagnosis; a first subset of target indicators that—when combined—support the first diagnosis; a second subset of target indicators that—when combined—support the first diagnosis; a first treatment pathway associated with treatment of the first diagnosis when supported by the first subset of target indicators; and a second treatment pathway associated with treatment of the first diagnosis when supported by the second subset of target indicators.

Additionally or alternatively, in one implementation, the computer system can implement a diagnostic module defining one or more treatment pathways for each distinct type of a particular diagnosis. In particular, in this implementation, as described above, the computer system can implement a diagnostic model defining a set of submodules linked to and/or integrated within each module, in the population of modules, defining a particular diagnosis, such that each submodule, in the set of submodules, defines a particular type of the particular diagnosis. For example, in this implementation, the computer system can implement a diagnostic module including a first module, in a population of modules, defining a first diagnosis (e.g., Diabetes) and including: a first submodule defining a first type (e.g., (e.g., Type I Diabetes) of the first diagnosis, a first set of target indicators supporting the first diagnosis of the first type, and a first set of treatment pathways associated with the treatment of the first diagnosis of the first type; and a second submodule defining a second type (e.g., Type II Diabetes) of the first diagnosis, a second set of target indicators supporting the first diagnosis of the second type, and a second set of treatment pathways associated with treatment of the first diagnosis of the second type.

### 4.3 Target Test Method: Missing Supporting Evidence

Additionally or alternatively, in another implementation, the computer system can implement a diagnostic model defining a target test method—such as a vital sign(s) check, a lab test (e.g., a WBC test, a culture test), an imaging exam (e.g., a CT scan, an X-ray exam), a patient survey, etc.—for obtaining health data associated with one or more target indicators defined for a particular module in the population of modules stored within the diagnostic model.

In particular, in this implementation, the computer system can implement a diagnostic model including a first module, in a population of modules, defining: a first diagnosis; a first set of target indicators supporting the first diagnosis; and, for a subset of target indicators (e.g., one or more target indicators), in the first set of target indicators, a first set of target test methods configured to yield health data associated with the subset of target indicators. For example, the first module can define: an atrial fibrillation diagnosis; a target indicator of a CHA₂DS₂-VASc score exceeding a threshold score and derived from patient health data including instances of prior stroke(s), patient diagnoses of diabetes, hypertension, heart failure, and/or vascular disease, patient age, patient sex, etc.; and a first set of target test methods configured to yield patient data associated with the CHA₂DS₂-VASc score, such as including a test method(s) associated with monitoring diabetes, hypertension, heart failure, and/or vascular disease in the patient. In this implementation, each other module, in the population of modules, can similarly define: a diagnosis; a set of target indicators supporting the diagnosis; and a set of target test methods configured to yield health data associated with the set of target indicators.

### 4.4 Linked Modules: Distinct Diagnoses

In one variation, the computer system can implement a diagnostic model linking modules corresponding to related and/or adjacent diagnoses defining similar target indicators. For example, the diagnostic model can define: a first module defining a first diagnosis defining a first set of target indicators including a first target indicator, a second target indicator, and a third target indicator; and a second module—linked to the first module—defining a second diagnosis defining a second set of target indicators including the first target indicator, the second target indicator, the third target indicator, and a fourth target indicator. In this example, the diagnostic model can link the second module to the first module and define a differentiating indicator for this module pair (i.e., the first and second module) corresponding to presence and/or absence of the fourth target indicator (e.g., in health data contained in an EHR of a patient). Furthermore, the diagnostic model can define a target test method—linked to the first and second modules—configured to yield health data indicative of presence and/or absence of the fourth target indicator.

### 5. Real-Time Note Augmentation

In one implementation, the computer system can leverage the diagnostic module to selectively suggest additional indicators for specifying in a note based on a diagnosis supplied by the provider. In particular, in this implementation, the computer system can: receive identification of a diagnosis from a provider (e.g., via the provider portal) for a patient; retrieve the diagnostic model to access a module, in the population of modules, corresponding to the diagnosis and defining a set of target indicators supporting the diagnosis; retrieve an electronic health record (or “EHR”) of the patient specified by the provider; and scan contents of the EHR for indicators corresponding to the set of target indicators. Then, in response to a first set of patient indicators (e.g., a measured heart rate, a measured blood pressure, a current lab result, a current age or weight, a prior diagnosis)—represented in the EHR—corresponding to the set of target indicators, the computer system can selectively present one or more indicators, in the first set of patient indicators, to the provider at the provider portal. The provider may then select and/or reject each of these suggested indicators for including and/or omitting in the note generated for a current patient encounter. In response to selection of a subset of indicators, in the set of indicators, the computer system can automatically append the note with the subset of indicators linked to the diagnosis.

The computer system can therefore enable (near) real-time augmentation of notes initiated by the provider by rapidly: identifying relevant patient data (i.e., indicators) obtained for the patient and supporting the provider-supplied diagnosis; and selectively surfacing this information to the provider. For example, the computer system can: receive a pneumonia diagnosis for a particular patient—and for a (current) encounter—from the provider (e.g., via the provider portal); and retrieve a pneumonia module—stored in the diagnostic model—and defining a first set of target indicators including: a target chest image (e.g., depicting positive indication of infiltrate, consolidation, density, or opacity within the patient's chest), a target body temperature exceeding a threshold temperature, and/or a target WBC exceeding a threshold WBC. The computer system can then: access an EHR of the patient containing a timeseries of health data collected for the patient during the current health encounter and/or over a preceding time period; and scan the timeseries of health data in the health record for presence of indicators corresponding to the set of target indicators.

In particular, the computer system can: locate a chest image—captured for the patient during the current encounter—depicting positive indication of infiltrate; and, in response to the chest image corresponding to the target chest image, flag the chest image as a first indicator for the pneumonia diagnosis. The computer system can further: locate a body temperature recorded for the patient during the current encounter and flag the body temperature as a second indicator for the pneumonia diagnosis in response to the body temperature exceeding the threshold temperature; and/or locate a WBC captured for the patient during the current encounter and withhold flagging the WBC as an indicator for the pneumonia diagnosis in response to the WBC falling below the threshold WBC. The computer system can then: compile the first and second indicators—corresponding to the positive chest image and the body temperature—into a list of indicators supporting the pneumonia diagnosis; and present this list of indicators to the provider—such as below the pneumonia diagnosis (e.g., entered by the provider)—within the provider portal. Then, in response to selection of the first indicator (e.g., the positive chest image) by the provider, the computer system can automatically append the note—initiated for the current encounter with the patient and specifying the pneumonia diagnosis—with the first indicator, such that the resulting note indicates the pneumonia diagnosis and collection of the positive chest image during the current encounter. Furthermore, in response to selection of the second indicator (e.g., the body temperature) by the provider, the computer system can additionally append the note with the second indicator, such that the note further indicates recordation of the patient's body temperature during the current encounter. Alternatively, in response to rejection of the second indicator by the provider, the computer system can withhold appending of the note with this second indicator.

### 5.1 Receiving a Diagnosis

Block S110 of the method S100 recites receiving identification of a first diagnosis, in a population of diagnoses, for a first encounter with a first patient from an instance of a provider portal accessed by a provider. In particular, the computer system can interface with the provider portal to receive a diagnosis for a particular patient from the patient's provider, such as during and/or succeeding an encounter with this particular patient.

For example, the provider may access the provider portal and select a patient profile associated with the patient, thereby triggering initiation of an encounter between the patient and this provider. In response to initiation of the encounter, the computer system can receive a set of encounter data, such as including: a patient identifier associated with the patient; a provider identifier associated with the provider; an initial timestamp corresponding to a start time of the encounter and/or a final timestamp corresponding to an end time of the encounter; and/or a passcode (e.g., configured to enable access to patient data—such as stored in a secure database—associated with the patient). Then, during the encounter, the provider may enter a particular diagnosis (e.g., pneumonia, heart disease, hypertension)—at the patient profile—for the patient within the provider portal. The computer system can then: receive this particular diagnosis for the patient for this encounter; and leverage the set of encounter data to retrieve patient indicators—extracted from patient data stored in the patient's health record—supporting this particular diagnosis, as further described below.

### 5.2 Diagnosis+Retrieving Target Indicators

Block S120 of the method S100 recites accessing a diagnostic model including a population of modules corresponding to a population of diagnoses, each module, in the population of modules, defining a set of target indicators supporting a corresponding diagnosis in the population of diagnoses. Furthermore, Block S122 of the method S100 recites accessing a first set of target indicators defined for the first diagnosis in a first module, in the population of modules, corresponding to the first diagnosis.

In particular, in response to receiving a particular diagnosis for a patient from the provider (e.g., via the provider portal), the computer system can: retrieve the diagnostic model and access a module, in the population of modules contained in the diagnostic model, corresponding to the particular diagnosis; and identify a set of target indicators defined in the module for the particular diagnosis. For example, in response to receiving a diagnosis of “pneumonia” from the provider, the computer system can: retrieve a module, in the population of modules of the diagnostic model, corresponding to the “pneumonia” diagnosis; and extract a set of target indicators including a target chest image (e.g., depicting positive indication of infiltrate, consolidation, density, or opacity within the patient's chest), and a body temperature exceeding a body temperature threshold.

### 5.2 Scanning the Patient Health Record+Patient Indicator Extraction

Block S130 of the method S100 recites: accessing a health record, in a population of health records, corresponding to the first patient and including a corpus of patient data associated with the first patient. Furthermore, Block S140 of the method S100 recites: extracting a first set of patient indicators, from the corpus of patient data, corresponding to the first set of target indicators.

In particular, in response to receiving the diagnosis from the provider (e.g., via the provider portal), the computer system can: retrieve the module, in the population of modules in the diagnostic model, corresponding to the diagnosis, and extract a set of target indicators defined for the diagnosis accordingly; retrieve a health record (e.g., an EHR) including timeseries health data recorded for the patient over time; scan the timeseries health data for presence of indicators corresponding to the set of target indicators; and therefore extract a set of identifiers—represented in timeseries health data recorded in the patient's health record—that correspond to the set of target identifiers defined for the particular diagnosis.

For example, in response to receiving a diagnosis of “pneumonia” for the patient from the provider (e.g., via the provider portal), the computer system can: retrieve the “pneumonia” module from the diagnostic model and extract a set of target indicators—including a target chest image (e.g., depicting positive indication of infiltrate, consolidation, density, or opacity within the patient's chest) and a body temperature exceeding a body temperature threshold—defined for the “pneumonia” diagnosis accordingly; access a health record—including a corpus of patient data (e.g., lab data, vitals data, imaging data, demographic data, lifestyle data)—associated with the patient; scan this corpus of patient data for patient indicators corresponding to the set of target indicators defined for the “pneumonia” diagnosis; and thus extract a set of patient indicators—such as including a positive chest image captured for the patient and/or a body temperature exceeding the body temperature threshold—that supports the diagnosis of “pneumonia” provided by the provider and corresponds to the set of target indicators.

In one variation, the computer system can selectively scan the health record based on a set of module parameters—such as defining specific combinations of target indicators that support the diagnosis, a set of rankings and/or evidence tiers (e.g., primary, secondary, tertiary), a set of target sampling windows, etc.—defined within the module (e.g., as described above). In one example, in response to the module defining a first subset of target indicators assigned a first rank and a second subset of target indicators assigned a second rank falling below the first rank, the computer system can: initially scan the health record for presence of indicators corresponding to the first subset of target indicators; and only scan the health record for presence of indicators corresponding to the second subset of target indicators in response to absence of any indicators corresponding to the first subset of target indicators in the health record. Alternatively, the computer system can scan the health record for presence of indicators corresponding to the second subset of target indicators in response to a quantity of indicators corresponding to the first subset of target indicators falling below a threshold quantity.

In another variation, the computer system can selectively filter health data contained in the health record based on time values (e.g., timestamps) linked to these health data. In particular, in one example, in response to the module defining a target indicator assigned a target sampling window of 24 hours, the computer system can: filter the timeseries of health data contained in the health record by timestamp to isolate a first timeseries of patient data captured within a 24-hour time period preceding a current encounter with the patient; and scan the first timeseries of health data for presence of an indicator corresponding to the target indicator. In another example, the module can define a target indicator supporting the diagnosis and defining: a first target sampling window of 24 hours assigned a first rank; and a second target sampling window of 72 hours assigned a second rank. In this example, the computer system can: filter the timeseries of health data by the timestamp to isolate a first timeseries of health data captured within a 24-hour time period preceding a current encounter; and scan the first timeseries of health data for presence of indicators corresponding to the target indicator. Then, in response to absence of an indicator corresponding to the target indicator in the first timeseries of health data, the computer system can: isolate a second timeseries of health data captured within a 72-hour time period preceding the current encounter; and scan the second timeseries of health data for presence of indicators corresponding to the target indicator.

### 5.4 Presenting Supporting Evidence

Block S160 of the method S100 recites: generating a notification including the first set of target indicators and a first prompt to review each patient indicator, in the first set of patient indicators, for specifying in the first provider note generated for the first encounter; and indicating the first diagnosis for the first patient transmitting a notification to the provider via the provider portal including a set of patient indicators, linked to a diagnosis, for specifying in a provider note generated for an encounter and indicating the diagnosis for the patient. In particular, in response to identifying a set of indicators—represented in the patient's health record and corresponding to the set of target indicators supporting the diagnosis—the computer system can selectively present the set of indicators to the provider within the provider portal.

For example, during a patient encounter for a patient, the provider may input a diagnosis for the patient via the provider portal. The computer system can then: execute the methods and techniques described above to extract a set of indicators from the patient's health record that support the diagnosis; and present the set of indicators in a list—such as rendered adjacent the diagnosis entered by the provider—within the provider portal in (near) real-time.

Additionally or alternatively, the computer system can selectively filter the set of indicators presented to the provider in order to: prioritize surfacing of key information supporting the provider-supplied diagnosis; limit overloading of the provider with nonessential information; and promote inclusion of key information—that best supports the provider's diagnosis—in the note and thereby increase likelihood of note acceptance and/or decrease risk of note rejection. For example, the computer system can: initially present a first subset of indicators to the provider for review, such as based on a set of module parameters defined for the module corresponding to the diagnosis; and, in response to selection of a quantity of indicators, in the first subset of indicators, exceeding a threshold quantity, append the note to include the quantity of indicators selected by the provider and withhold presentation of additional indicators to the provider. Alternatively, in response to the quantity of indicators selected by the provider falling below the threshold quantity, the computer system can automatically present a second subset of indicators to the provider for review, such as in replacement of unselected and/or rejected indicators in the first subset of indicators. The computer system can therefore selectively surface additional indicators responsive to selections and/or feedback from the provider to promote including of at least the threshold quantity of indicators in the note.

In one implementation, the computer system can: access a target quantity of indicators—such as corresponding to a threshold confidence (e.g., 50%, 70%, 90%, 99%) of note acceptance—defined by the module corresponding to the diagnosis; and select a subset of indicators—corresponding to the target quantity—from the set of indicators extracted from the health record for presenting to the provider within the provider portal.

### 5.4.1 Variation: Target Indicator Rank

In one variation, the computer system can order the set of indicators within the list based on a ranking or weight assigned to each indicator in the set of indicators. For example, the computer system can: arrange a first indicator—corresponding to a first target indicator defining a first rank—within a first position in the list (e.g., at a top of the list); arrange a second indicator—corresponding to a second target indicator defining a second rank—within a second position, below the first position, in the list; and arrange a third indicator—corresponding to a third target indicator defining a third rank—within a third position (e.g., at a bottom of the list), below the second position, in the list.

In one example, during a patient encounter for a patient, the provider may input an anemia diagnosis for the patient (e.g., via the provider portal). In this example, in response to receiving the anemia diagnosis, the computer system can extract a set of target indicators from an “anemia” module, in the population of modules, corresponding to the anemia diagnosis, such as including: a first target indicator—defining a first rank—corresponding to a hemoglobin level less than a hemoglobin threshold; a second target indicator—defining a second rank less than the first rank—corresponding to a hematocrit level less than a hematocrit threshold; and a third target indicator—defining a third rank less than the second rank—corresponding to a mean corpuscular volume (hereinafter “MCV”) reading within an MCV range. The computer system can then execute the methods and techniques described above to extract a set of patient indicators (or “indicators”) from the patient's health record that support the anemia diagnosis. In particular, in this example, the computer system can extract: a first patient indicator corresponding to a hemoglobin level recorded for the patient and falling below the hemoglobin threshold; a second patient indicator corresponding to a hematocrit level recorded for the patient and falling below the hematocrit threshold; and a third patient indicator corresponding to an MCV reading recorded for the patient and falling within the MCV range. The computer system can then: generate a notification including the first, second, and third patient indicators presented in order based on the first, second, and third ranks, such that the first patient indicator is located in a first slot, the second patient indicator is located in a second slot following the first slot (e.g., below and/or adjacent the first slot), and the third patient indicator is located in a third slot following the second slot; and transmit the notification to the provider (e.g., via the provider portal). Therefore, the computer system can selectively present the set of indicators to the provider by ordering the set of indicators within the list based on a ranking or weight assigned to each indicator in the set of indicators, thereby promoting review and/or selection of evidence—assigned a greater weight or higher rank—by the provider for pairing with the diagnosis in a provider note generated for the encounter.

### 5.4.2 Variation: Target Indicator Tier

In another variation, the computer system can selectively present a subset of indicators, in the set of indicators, to the provider based on a set of evidence tiers defined by the module corresponding to the diagnosis. For example, the module can define: a set of primary target indicators; a set of secondary target indicators; and a set of tertiary target indicators. The computer system can then extract a set of indicators—corresponding to target indicators in the first, second, and/or third set of target indicators—from the health record and selectively: present a first subset of indicators corresponding to the set of primary target indicators in response to a first quantity of indicators in the first subset of indicators exceeding a threshold quantity; present a second subset of indicators corresponding to target indicators in the set of primary and secondary target indicators in response to the first quantity falling below the threshold quantity and a second quantity of indicators, in the second subset of indicators, exceeding the threshold quantity; and present a third subset of indicators corresponding to target indicators in the set of primary, secondary, and tertiary target indicators in response to the second quantity falling below the threshold quantity. In this example, the computer system can therefore prioritize presentation of primary indicators and selectively present secondary and/or tertiary indicators as required.

In one example, in response to receiving an “anemia” diagnosis for the patient from the provider (e.g., via the provider portal), the computer system can retrieve the “anemia” module from the diagnostic model and extract a set of target indicators including: a set of primary target indicators—supporting the anemia diagnosis and required for predicting the anemia diagnosis—including a first primary target indicator corresponding to a hemoglobin level falling below a hemoglobin threshold and a second primary target indicator corresponding to a hematocrit level falling below a hematocrit threshold; and a set of secondary target indicators—supporting the anemia diagnosis in combination with the set of primary target indicators—including a first secondary target indicator corresponding to a vitamin D level falling below a vitamin D threshold and a second secondary target indicator corresponding to a vitamin B12 level falling below a vitamin B12 threshold. The computer system can then: execute the methods and techniques described above to: extract a first primary patient indicator—corresponding to a hemoglobin level recorded for the patient and falling below the hemoglobin threshold—from the patient's health record; extract a second primary patient indicator—corresponding to a hematocrit level recorded for the patient and falling below the hematocrit threshold—from the patient's health record; and, in response to a quantity of two primary patient indicators exceeding a threshold quantity of primary indicators (e.g., defined for the anemia module), the computer system can present the first and second primary patient indicators to the provider (e.g., via notification within the provider portal) in support of the “anemia” diagnosis.

Alternatively, in the preceding example, in response to the quantity of primary patient indicators falling below the threshold quantity and/or in response to absence of the first and/or second primary patient indicators in the patient health record, the computer system can: extract a first secondary patient indicator—corresponding to a vitamin D level recorded for the patient—falling below the vitamin D threshold; extract a second secondary patient indicator corresponding to a vitamin B12 level recorded for the patient—falling below the vitamin B12 threshold; and present the first and second secondary patient indicators—such as in combination with the first and second primary patient indicators (e.g., if present in the patient health record) to the provider (e.g., via notification within the provider portal. Therefore, the computer system can prioritize presentation of primary indicators—and selectively present secondary and/or tertiary indicators—to the provider in support of diagnoses.

### 5.4.3 Variation: Provider Preferences

In one variation, the computer system can leverage a set of preferences defined for a particular entity (e.g., a provider, a hospital, an insurance company) to selectively filter and/or present a set of indicators—for pairing with a particular diagnosis—within the provider portal. In particular, in this variation, the computer system can: store a set of preferences—such as including a minimum quantity of indicators, a maximum quantity of indicators, a preferred indicator or indicators, a preferred combination of indicators, an unfavored indicator or indicators, a preferred sampling window, etc.—in an entity profile affiliated with a particular entity; and leverage the set of preferences to selectively present indicators—extracted from patient health records—within the provider portal in support of a diagnosis.

In one implementation, the computer system can: learn and/or store a set of provider preferences in a provider profile affiliated with the provider; and leverage the provider preferences to select specific indicators—which the provider may be more likely to consider and/or select for specifying in a provider note—for presenting to the provider in support of diagnoses. For example, during an initial time period, for each diagnosis entered by the provider, the computer system can: implement methods and techniques described above to selectively present the provider with a set of indicators—extracted from patient health data—supporting the diagnosis; receive selection of a first subset of indicators, in the set of indicators, for specifying in a provider note; generate a data packet—including the diagnosis, the first subset of indicators labelled as “selected,” a second subset of indicators labelled as “unselected,” a quantity of indicators selected (e.g., in the first subset of indicators), etc.—representing the diagnosis; and insert the data packet in a library of data packets stored in the provider profile. The computer system can then leverage this library of data packets to “learn” or derive a set of provider preferences, such as across all diagnoses and/or for a particular diagnosis.

In particular, in this variation, Blocks of the method S100 can recite: generating a data packet including the indicators selected and/or unselected by the provider in Block S174; and storing the data packet in a library of data packets linked to a provider profile in Block S184.

In one example, in response to receiving an anemia diagnosis for the patient from the provider (e.g., via the provider portal), the computer system can execute the methods and techniques described above to extract a set of patient indicators from the patient's health record—supporting the anemia diagnosis and corresponding to a set of target indicators defined in a corresponding module of the diagnostic model—including: a first patient indicator corresponding to a hemoglobin level falling below a hemoglobin threshold (e.g., defined in the module); and a second patient indicator corresponding to a hematocrit level falling below a hematocrit threshold (e.g., defined in the module). The computer system can then: present the first and second patient indicator to the provider (e.g., via the provider portal) in support of the anemia diagnosis; and, in response to receiving selection of the first patient indicator (e.g., from the provider) for specifying in a provider note specifying the anemia diagnosis generate a data packet including the anemia diagnosis, the first patient indicator (e.g., selected by the provider), and the second patient indicator (e.g., unselected by the provider), label the first patient indicator—stored in the data packet—with a first value representing selection of the first patient indicator by the provider, label the second patient indicator—stored in the data packet—with a second value representing rejection of the second patient indicator by the provider, and store the data packet in a library of data packets linked to a provider profile, in a population of provider profiles, associated with the provider.

Later, during a subsequent encounter with a second patient, in response to receiving an anemia diagnosis for the second patient, the computer system can: extract a second set of patient indicators—supporting the anemia diagnosis—from the second patient's health record; and, based on the library of data packets, select a subset of patient indicators, in the second set of patient indicators, for presenting to the provider in support of the anemia diagnosis. In particular, in this example, the computer system can prioritize presentation of hemoglobin levels—such as in replacement of other patient indicators and/or in a first position in a list of patient indicators—in support of anemia diagnoses entered by the provider, based on selection of the first patient indicator (e.g., a hemoglobin level) as indicated in the data packet. The computer system can therefore prioritize presentation of indicators historically preferred by the provider for a particular diagnosis and/or derived from historical data stored in the provider profile, thereby increasing likelihood of selection of these patient indicators by the provider.

### 5.4.4 Variation: Insurer Preferences

In another variation, the computer system can: learn and/or store a set of insurer preferences—such as for a particular insurance company (e.g., medical insurance company)—in an insurer profile affiliated with the insurance company; and leverage the insurer preferences to select specific indicators—which may be more likely to yield acceptance of provider notes specifying these indicators—for presenting to the provider in support of diagnoses. For example, during an initial time period, for each provider note, in a corpus of provider notes, sent to the insurer, the computer system can: initialize a data packet corresponding to the provider note; record a diagnosis and a set of indicators specified in the provider note to the data packet; receive confirmation of acceptance or rejection of the provider by the insurer and label the data packet accordingly; and insert the data packet in a library of data packets stored in the insurer profile. The computer system can then leverage this library of data packets to “learn” or derive a set of insurer preferences—such as including quantities and/or combinations of indicators across all diagnoses and/or for a particular diagnosis—most likely to yield acceptance of provider notes.

In one implementation, Blocks of the method S100 can include: transmitting the provider note to an insurance company affiliated with the first patient in Block S182; receiving confirmation of acceptance of the provider note by the insurance company and labeling the data packet as accepted in Block S186; and inserting the data packet in a library of data packets stored in an insurer profile in Block S188.

In one example, during a first patient encounter for a first patient, the provider can input an anemia diagnosis for the first patient (e.g., via the provider portal). Then, in response to receiving selection of a first subset of patient indicators supporting the anemia diagnosis, from the provider (e.g., via the provider portal), for specifying in a first provider note, the computer system can: generate a first data packet corresponding to the first provider note; and record the anemia diagnosis and the first subset of patient indicators—specified in the first provider note—to the first data packet. In particular, in this example, the first subset of patient indicators can include: a first patient indicator—corresponding to a hemoglobin level recorded for the first patient—falling below the hemoglobin threshold; and a second patient indicator—corresponding to a hematocrit level recorded for the first patient—falling below the hematocrit threshold. The computer system can then: receive confirmation of acceptance of the first provider note by the insurance company affiliated with the first patient; label the first data packet as accepted; and insert first the data packet in a library of data packets stored in an insurer profile. Then, during a second patient encounter for a second patient, the provider may input an anemia diagnosis for the second patient (e.g., via the provider portal). In particular, in this example, based on the library of data packets stored in an insurer profile, the computer system can select a second subset of patient indicators, in a second set of patient indicators supporting the anemia diagnosis for the second patient, for presenting to the provider. Therefore, the computer system can prioritize presentation of preferred indicators for a particular diagnosis derived from an insurer profile.

Additionally or alternatively, in one variation, the computer system can prompt the entity—such as the provider or an administrator—to manually populate a set of preferences.

### 5.5 Generating a Provider Note

Generally, as described above, the computer system can host or interface with the provider portal—accessed by the provider—to aid the provider in producing a provider note (e.g., an electronic progress note, an electronic clinical note) containing relevant information for a corresponding encounter (or “patient encounter”) between a patient and the provider. In one implementation, the computer system can compile a set of provider note data including: the diagnosis; a set of patient indicators supporting a diagnosis and selected by a provider; and a treatment pathway, in a set of treatment pathways, selected by the provider (e.g., via the provider portal). For example, in response to receiving selection of a subset patient indicators—from a set of patient indicators supporting a pneumonia diagnosis supplied by the provider—from the provider, the computer system can compile a set of provider note data including: the pneumonia diagnosis; the subset of patient indicators selected by the provider and supporting the pneumonia diagnosis; and/or a treatment pathway—in a set of treatment pathways defined for the pneumonia diagnosis—selected and/or provided by the provider. The computer system can then leverage the set of provider note data to generate and/or populate a provider note for the encounter with the patient.

### 5.6 Provider Note: Predicting an Acceptance Score

In one implementation, Block S170 recites: predicting a first acceptance score for the first provider note based on the first diagnosis and the first subset of patient indicators, the first acceptance score representing a likelihood of acceptance of the first diagnosis for the first patient during the first encounter represented by the first provider note.

In particular, in this implementation, the computer system can predict an acceptance score for a provider note—indicating a particular diagnosis for a particular encounter with a patient—based on the particular diagnosis and patient indicators selected by the provider for specifying in the provider note. For example, the computer system can predict an acceptance score—representing a likelihood of acceptance of the provider note by an insurance company affiliated with the patient—for a provider note generated for an encounter with a patient. In particular, in one example, the computer system can predict an acceptance score represented by a percentage, a score between 1 and 10, a qualitative score (e.g., low, high, possible, probable), a binary score, etc. The computer system can compare the acceptance score to a threshold score—defined in the diagnostic model—to predict acceptance and/or rejection of a particular provider note.

In one implementation, the computer system can implement a single threshold score (e.g., a uniform threshold score) for each module in the population of modules. For example, the computer system can access a threshold score of 80%, defined for each module, in the population of modules, required to predict a diagnosis. Alternatively, in another implementation, the computer system can implement a variable threshold score defined for each individual module in the population of modules. For example, the computer system can: access a threshold score of 80%, defined for a first module and required to predict a first diagnosis; and access a threshold score of 50%, defined for a second module and required to predict a second diagnosis.

In particular, in this implementation, the computer system can: receive a diagnosis from the provider (e.g., via the provider portal) for a patient for an encounter; extract a set of patient indicators—from a corpus of patient data associated with the patient—corresponding to the set of target indicators supporting the diagnosis; and transmit a notification to the provider (e.g., via the provider portal), the notification including a prompt to review each patient indicator, in the set of patient indicators, for specifying in the provider note generated for the encounter and indicating the diagnosis for the patient. Then, in response to receiving selection of a subset of patient indicators—from the set of patient indicators—from the provider (e.g., via the provider portal), for specifying in the provider note generated for the encounter, the computer system can: append the provider note with the subset of patient indicators linked to the diagnosis; and, based on a subset of patient indicators selected by the provider and the particular diagnosis, predict an acceptance score for the provider note. For example, in response to the provider selecting a first subset of patient indicators—such as including a first quantity of patient indicators and/or a first combination of patient indicators—the computer system can predict a first acceptance score. Alternatively, in response to the provider selecting a second subset of patient indicators—such as including a second quantity of patient indicators and/or a second combination of patient indicators—the computer system can predict a second acceptance score. Therefore, the computer system can selectively predict the acceptance score for the provider note based on identification of patient indicators—from the health record—corresponding to the set of target indicators defined for a diagnosis, thereby: reducing costs to the patient due to an unaccepted provider note; and reducing resources dedicated in generating the provider note and/or implementing post-hoc corrections due to an unaccepted provider note.

### 5.6.1 Acceptance Score: Quantity of Patient Indicators

In in one implementation, the computer system can predict the acceptance score for the provider note generated for the encounter based on a quantity of patient indicators selected by the provider. In particular, in this implementation, the computer system can: receive a diagnosis from the provider (e.g., via the provider portal) for a patient for an encounter; extract a set of patient indicators—from a corpus of patient data associated with the patient—corresponding to the set of target indicators supporting the diagnosis; and transmit a notification to the provider (e.g., via the provider portal), the notification including a prompt to review each patient indicator, in the set of patient indicators, for specifying in the provider note generated for the encounter and indicating the diagnosis for the patient. Then, in response to receiving selection of a subset of patient indicators—from the set of patient indicators—from the provider (e.g., via the provider portal) for specifying in the provider note generated for the encounter, the computer system can: append the provider note with the subset of patient indicators linked to the diagnosis; access a quantity of patient indicators in the subset of patient indicators selected by the provider; and predict the acceptance score for the provider note based on the quantity of patient indicators.

For example, the computer system can receive a pneumonia diagnosis from the provider (e.g., via the provider portal) for a patient for an encounter. In particular, in this example, the computer system can access a pneumonia module defining a set of three target indicators supporting the pneumonia diagnosis including: a first target indicator corresponding to a target chest image (e.g., depicting positive indication of infiltrate, consolidation, density, or opacity within the patient's chest); a second target indicator corresponding to a body temperature exceeding a body temperature threshold; and a third target indicator corresponding to a white blood cell count (or “WBC”) exceeding a WBC threshold. For the encounter with the patient, the computer system can: scan a health record—associated with the patient—for the set of three target indicators defined for the pneumonia diagnosis; extract a first patient indicator—corresponding to a positive chest image recorded for the patient—depicting positive indication of infiltrate; and extract a second patient indicator—corresponding to a body temperature recorded for the patient—exceeding the body temperature threshold. The computer system can then transmit a notification to the provider (e.g., via the provider portal) including the first and second patient indicators and a prompt to review each patient indicator for specifying in the provider note—indicating the pneumonia diagnosis—generated for the encounter. In response to receiving selection of two patient indicators (e.g., the positive chest image and the body temperature)—from the list of two patient indicators presented to the provider—for specifying in the provider note, the computer system can then: append the provider note with the first and second patient indicators, such that the resulting provider note indicates the pneumonia diagnosis and collection of the positive chest image and the body temperature during the encounter; and, based on a quantity of two patient indicators selected for the provider note, predict an acceptance score of 80% for the provider note.

Alternatively, in the preceding example, in response to receiving selection of only the first patient indicator (e.g., the positive chest image)—from the first and second patient indicators transmitted to the provider—the computer system can: append the provider note with the first patient indicator, such that the resulting provider note indicates the pneumonia diagnosis and collection of the positive chest image during the encounter; and, based on a quantity of one patient indicator selected for the provider note, predict a second acceptance score—less than the first acceptance score—of 50% for the provider note. Therefore, the computer system can predict the acceptance score for the provider note based on the quantity of patient indicators—from the health record—corresponding to the set of target indicators.

### 5.6.2 Acceptance Score: Insurer Preferences

Additionally or alternatively, in one implementation, the computer system can predict the acceptance score for the provider note generated for the encounter based on a set of insurer preferences defined and/or derived for a particular diagnosis and an insurance company affiliated with the patient.

In particular, in this implementation, the computer system can implement methods and techniques described above to: selectively present a set of patient indicators—corresponding to target indicators defined for a diagnosis—to the provider in response to receiving the diagnosis for a patient from the provider; receive selection of a subset of patient indicators, in the set of patient indicators, for specifying in a provider note indicating the diagnosis; and generate and/or append the provider note with the subset of patient indicators linked to the diagnosis accordingly. The computer system can then: access a set of insurer preferences defined and/or derived for an insurance company affiliated with the patient and/or for the diagnosis, such as including a threshold quantity of patient indicators and/or a set of preferred patient indicators defined for the diagnosis; and derive an acceptance score for the provider note based on these insurer preferences and the subset of patient indicators included in the provider note. For example, the computer system can: characterize a first difference between a quantity of patient indicators, in the subset of patient indicators, and the threshold quantity of patient indicators; characterize a second difference between the subset of patient indicators and the set of preferred patient indicators; and, based on the first difference and the second difference, predict an acceptance score for the provider note specifying the subset of patient indicators.

For example, in response to receiving a pneumonia diagnosis for a patient, the computer system can implement methods and techniques described above to: extract a first patient indicator—corresponding to a positive chest image recorded for the patient depicting positive indication of infiltrate and supporting the pneumonia diagnosis—from the patient's health record; extract a second patient indicator—corresponding to a body temperature exceeding a body temperature threshold and supporting the pneumonia diagnosis—from the patient record; selectively present the first and second patient indicators to the provider via the provider portal; and, in response to receiving selection of both the first and second patient indicators from the provider, append a provider note—indicating the pneumonia diagnosis for the patient—with the first and second patient indicators, such that the resulting provider note indicates the pneumonia diagnosis and collection of the positive chest image and the body temperature during the encounter. In order to predict an acceptance score for the provider note, the computer system can then: access a threshold quantity of two patient indicators defined for the pneumonia diagnosis by an insurance company affiliated with the patient; access a set of preferred target indicators defined for the pneumonia diagnosis by the insurance company, such as including a first preferred target indicator corresponding to a target chest image (e.g., depicting positive indication of infiltrate, consolidation, density, or opacity within the patient's chest) and a second preferred target indicator corresponding to a respiratory rate exceeding a respiratory rate threshold; characterize a first difference between a quantity of patient indicators included in the provider note and the threshold quantity; characterize a second difference between the first and second patient indicator and the set of preferred target indicators; and—based on the first difference and the second difference—predict a first acceptance score of 80% for the provider note.

Alternatively, in the preceding example, in response to receiving selection of only the first patient indicator (e.g., the positive chest image)—from the first and second patient indicators transmitted to the provider—from the provider, the computer system can: append the provider note with the first patient indicator, such that the resulting provider note indicates the pneumonia diagnosis and collection of the positive chest image during the encounter; characterize a third difference between the quantity of patient indicators and the threshold quantity; characterize a fourth difference between the first patient indicator and the set of preferred patient indicators; and predict a second acceptance score—less than the first acceptance score—of 20% for the provider note. Therefore, the computer system can: predict the acceptance score for the provider note for the patient based on the quantity of patient indicators—from the health record and selected by the provider—corresponding to the set of target indicators meeting the threshold quantity indicators and the presence of preferred indicators, defined and/or derived for the insurance company, thereby increasing a likelihood of acceptance of the provider note by relying on insurer preferences to predict the acceptance score.

### 5.6.3 Acceptance Score: Primary & Secondary Indicators

Additionally or alternatively, in one implementation, the computer system can: predict the acceptance score for the provider note generated for the encounter based on presence of primary and secondary patient indicators supporting a diagnosis. In particular, in this implementation, the computer system can: receive a diagnosis from the provider (e.g., via the provider portal) for a patient for an encounter; and access a set of target indicators—defined in a module corresponding to the diagnosis—including a set of primary target indicators supporting and required for predicting the diagnosis and a set of secondary target indicators supporting the diagnosis in combination with the set of primary target indicators. The computer system can then extract a first subset of primary patient indicators—from a corpus of patient data associated with the patient—corresponding to the set of primary target indicators. In response to detecting a quantity of patient indicators in the first subset of primary patient indicators exceeding a threshold quantity, the computer system can transmit a notification to the provider (e.g., via the provider portal) including the set of primary target indicators and a prompt to review each patient indicator, in the first subset of primary patient indicators, for specifying in the provider note generated for the encounter and indicating the diagnosis for the patient. In particular, in this implementation, in response to receiving selection of a second subset of primary patient indicators—from the first subset of primary patient indicators—from the provider (e.g., via the provider portal), for specifying in the provider note generated for the encounter, the computer system can: append the provider note with the second subset of primary patient indicators linked to the diagnosis; and predict the acceptance score for the provider note based on the diagnosis and the second subset of primary patient indicators.

For example, the computer system can: receive a pneumonia diagnosis from the provider (e.g., via the provider portal) for a patient for an encounter; access a pneumonia module—in the population of modules contained in the diagnostic model—corresponding to the pneumonia diagnosis; and access a set of target indicators defined for the pneumonia diagnosis. In particular, in this example, the computer system can access a pneumonia module defining a set of primary target indicators supporting the pneumonia diagnosis and required for predicting the pneumonia diagnosis including: a first primary target indicator corresponding to a target chest image (e.g., depicting positive indication of infiltrate, consolidation, density, or opacity within the patient's chest); and a second primary target indicator corresponding to a body temperature exceeding a body temperature threshold. Furthermore, in this example, the computer system can access a set of secondary target indicators supporting the pneumonia diagnosis in combination with the set of primary target indicators including: a first secondary target indicator corresponding to a white blood cell count (or “WBC”) exceeding a WBC threshold; and a second secondary target indicator corresponding to a respiratory rate exceeding a respiratory rate threshold.

For the encounter with the patient, the computer system can: scan a health record—associated with the patient—for the set of primary and secondary target indicators defined for the pneumonia diagnosis; extract a first primary patient indicator—corresponding to a positive chest image recorded for the patient—depicting positive indication of infiltrate; and extract a second primary patient indicator—corresponding to a body temperature recorded for the patient—exceeding the body temperature threshold. The computer system can then: detect a first quantity of two primary patient indicators—in the health record associated with the patient—supporting the pneumonia diagnosis; and, in response to the first quantity of primary patient indicators exceeding a first threshold quantity, transmit a notification to the provider (e.g., via the provider portal) including the first and second primary patient indicators and a prompt to review each patient indicator for specifying in the provider note generated for the encounter. In particular, in this example, in response to receiving selection of the two primary patient indicators (e.g., the positive chest image and the body temperature)—from the list of two primary patient indicators supporting the pneumonia diagnosis—from the provider (e.g., via the provider portal), the computer system can: append the provider note with the first and second primary patient indicators, such that the resulting provider note indicates the pneumonia diagnosis and collection of the positive chest image and the body temperature during the encounter; and predict a first acceptance score—based on the presence of two primary patient indicators—of 100% for the provider note.

Alternatively, in the preceding example, in response to receiving selection of only the first patient indicator (e.g., the positive chest image)—from the first and second primary patient indicators transmitted to the provider—from the provider, the computer system can: append the provider note with the first primary patient indicator, such that the resulting provider note indicates the pneumonia diagnosis and collection of the positive chest image during the encounter; and predict a second acceptance score—less than the first acceptance score and based on the presence of one primary patient indicator—of 80% for the provider note.

Alternatively, in the preceding example, in response to absence of the first and second primary patient indicators—in the health record associated with the patient—the computer system can: extract a first secondary patient indicator—corresponding to a WBC recorded for the patient—exceeding the WBC threshold; and extract a second secondary patient indicator—corresponding to a respiratory rate recorded for the patient—exceeding the respiratory rate threshold. The computer system can then: detect a second quantity of two secondary patient indicators—in the health record associated with the patient—supporting the pneumonia diagnosis; and, in response to the second quantity of secondary patient indicators exceeding a second threshold quantity, transmit a notification to the provider (e.g., via the provider portal) including the first and second secondary patient indicators and a prompt to review each patient indicator for specifying in the provider note generated for the encounter and indicating the pneumonia diagnosis for the patient. In particular, in this example, in response to receiving selection of the two secondary patient indicators (e.g., the WBC and the respiratory rate)—from the list of two secondary patient indicators supporting the pneumonia diagnosis—from the provider (e.g., via the provider portal), for specifying in the provider note generated for the encounter, the computer system can: append the provider note with the first and second secondary patient indicators, such that the resulting provider note indicates the pneumonia diagnosis and collection of the WBC and the respiratory rate during the encounter; and predict a third acceptance score—less than the first and second acceptance scores and based on the presence of two secondary patient indicators—of 50% for the provider note. Therefore, the computer system can predict the acceptance score for the provider note generated for the encounter based on the presence of primary and secondary patient indicators—from the health record—corresponding to the set of primary and secondary target indicators.

### 5.6.4 Acceptance Score: Presence of Treatment Pathway

Additionally or alternatively, in one implementation, the computer system can predict the acceptance score for the provider note based on the presence of a treatment pathway selected by the provider. In particular, in this implementation, in response to receiving selection of a set of patient indicators—extracted from a health record associated with a patient and supporting a particular diagnosis—from the provider (e.g., via the provider portal), for specifying in the provider note generated for the encounter, the computer system can: append the provider note with the set of patient indicators linked to the diagnosis; characterize presence of a treatment pathway—in a set of treatment pathways defined for the diagnosis—included in the provider note; and predict the acceptance score based on the presence of the treatment pathway.

For example, the computer system can: receive selection of a set of patient indicators—from a health record associated with a patient and supporting a pneumonia diagnosis—from the provider (e.g., via the provider portal), for specifying in the provider note generated for the encounter; append the provider note with the set of patient indicators linked to the pneumonia diagnosis; identify a treatment pathway included in the provider note such as including a set of antibiotics and/or a set of medical procedures (e.g., intubation, extubation)—associated with treatment and/or mitigation of pneumonia; and predict a first acceptance score—based on the set of patient indicators and the presence of a treatment pathway selected by the provider—of 100% for the provider note. Alternatively, in the preceding example, in response to detecting absence of a treatment pathway included in the provider note, the computer system can predict a second acceptance score—based on the set of patient indicators and the absence of a treatment pathway selected by the provider—of 50% for the provider note. Therefore, the computer system can selectively predict the acceptance score for the provider note for the patient based on presence of a treatment pathway included in the provider note.

### 5.7 Verifying the Provider Note

Block S180 of the method S100 recites, in response to the first acceptance score exceeding a threshold score, verifying the first provider note. Additionally or alternatively, in one implementation, the computer system can prompt the provider to verify the provider note. For example, in this implementation, in response to the acceptance score exceeding the threshold score, the computer system can: compile a set of provider note data including the diagnosis, a set of patient indicators supporting a diagnosis and selected by a provider, and a treatment pathway, in a set of treatment pathways, selected by the provider; and transmit a notification—including a prompt to verify the provider note for transmitting to an insurance company affiliated with the patient—to the provider (e.g., via the provider portal).

### 5.8 Acceptance Score: Falling Below Threshold Score

Block S171 of the method S100 recites, in response to the acceptance score falling below the threshold score, withholding verification of the provider note.

In one variation, Block S161 of the method S100 recites transmitting a notification to the provider (e.g., via the provider portal) indicating the acceptance score falling below the threshold score and including a prompt to select additional patient indicators for specifying in the first provider note. In one implementation, in response to predicting an acceptance score falling below the threshold score, the computer system can withhold verification of a provider note for an encounter with a patient and suggest selecting additional patient indicators for specifying in the provider note. In particular, in this implementation, in response to receiving selection of a subset of patient indicators from the provider (e.g., via the provider portal) and predicting the acceptance score falling below the threshold score, the computer system can: withhold verification of the provider note for the encounter; and transmit a notification to the provider (e.g., via the provider portal) indicating the acceptance score falling below the threshold score and including a suggestion to select additional indicators from the set of patient indicators for specifying in the provider note.

For example, the computer system can receive a pneumonia diagnosis from the provider (e.g., via the provider portal) for a patient for an encounter. The computer system can implement the methods and techniques described above to extract a first patient indicator—corresponding to a positive chest image recorded for the patient—depicting positive indication of infiltrate; and extract a second patient indicator—corresponding to a body temperature recorded for the patient—exceeding the body temperature threshold. In particular, in this example, in response to receiving selection of the first patient indicator (e.g., the positive chest image)—from the list of two patient indicators supporting the pneumonia diagnosis—from the provider (e.g., via the provider portal), for specifying in the provider note generated for the encounter, the computer system can: append the provider note with the first patient indicator, such that the resulting provider note indicates the pneumonia diagnosis and collection of the positive chest image during the encounter; and predict a first acceptance score—based on the first patient indicator—of 20% for the provider note. In response to the first acceptance score of 20% falling below the threshold score of 50%, the computer system can: withhold verification of the provider note for the encounter; and transmit a second notification to the provider (e.g., via the provider portal) indicating the first acceptance score falling below the threshold score and including a suggestion to select additional indicators—from the list of two patient indicators supporting the pneumonia diagnosis—for specifying in the provider note. In response to receiving selection of the second patient indicator (e.g., the body temperature)—from the list of two patient indicators supporting the pneumonia diagnosis—from the provider (e.g., via the provider portal), for specifying in the provider note, the computer system can then: append the provider note with the second patient indicator, such that the resulting provider note indicates the pneumonia diagnosis and collection of the positive chest image and the body temperature during the encounter; and predict a second acceptance score of 80% for the provider note. Therefore, the computer system can selectively withhold verification of the provider note and/or prompt the provider to select additional patient indicators, thereby promoting an increase in acceptance of provider notes.

### 5.9 Insufficient Evidence to Support Provider Supplied Diagnosis

In one variation, the computer system can suggest retrieval of additional patient data to supplement a diagnosis supplied by the provider, such as in response to confidence in note acceptance falling below a threshold confidence. For example, the computer system can: generate a notification indicating insufficient evidence included in a note for a particular diagnosis and including a suggestion to execute a particular test—defined by a module, in the diagnostic model, corresponding to the particular diagnosis—configured to retrieve additional health data that may support the particular diagnosis.

In one implementation, the computer system can transmit a prompt to the provider suggesting retrieval of additional health data for the patient in response to a quantity of indicators—extracted from the health record of the patient and supporting a diagnosis—falling below a threshold quantity required for acceptance of a note specifying the diagnosis. For example, in response to receiving a diagnosis from a provider, the computer system can: retrieve a module, contained in the diagnostic model, corresponding to the diagnosis and defining a set of target indicators—including a first target indicator and a second target indicator—supporting the diagnosis; and scan the health record of the patient for indicators corresponding to the set of target indicators. Then, in response to identifying a first indicator corresponding to the first target indicator and detecting absence of a second indicator corresponding to the second target indicator, the computer system can: present the first indicator to the provider within the provider portal; access a target test method defined for the second target indicator within the module; generate a notification—indicating insufficient evidence for acceptance of a note specifying the diagnosis—including a suggestion to execute the target test method with the patient; and transmit the notification to the provider within the provider portal.

### 5.9.1 Insufficient Evidence: Missing Target Indicator

In one variation, Blocks of the method S100 can include: in response to detecting absence of a patient indicator—in the corpus of patient data associated with a patient—supporting a diagnosis, transmitting a notification to the provider (e.g., via the provider portal) of insufficient evidence in the health record to support the diagnosis and including a suggestion to execute a target test method for the patient in Block S166; accessing a health record associated with a patient including additional patient health data in Block S156; and transmitting a prompt to the provider via the provider portal including a prompt to select new patient indicators in Block S157. In particular, in this variation, the computer system can suggest retrieval of additional patient data to supplement a diagnosis supplied by the provider, such as in response to the corpus of patient data missing a target indicator.

In one implementation, the computer system can: receive a diagnosis from the provider (e.g., via the provider portal) for a patient for an encounter; and transmit a notification to the provider (e.g., via the provider portal) suggesting retrieval of additional health data for the patient in response to extracting a first target indicator and detecting absence of a second target indicator in a corpus of patient data associated with the patient. More specifically, in this implementation, the computer system can: access a target test method—configured to yield a patient indicator corresponding to the second target indicator—defined for the second target indicator within the module; and transmit the notification to the provider (e.g., via the provider portal) indicating insufficient evidence for the diagnosis and including a suggestion to execute the target test method with the patient.

For example, the computer system can receive an atrial fibrillation diagnosis from the provider (e.g., via the provider portal) for a patient for an encounter. The computer system can then scan a health record—associated with the patient—for a first target indicator (e.g., a CHA₂DS₂-VASc score exceeding a threshold score and derived from patient health data including instances of prior stroke(s), patient diagnoses of diabetes, hypertension, heart failure, and/or vascular disease, patient age, patient sex, etc.) and second target indicator (e.g., a target electrocardiogram (or “EKG”) reading depicting absence of P-waves, an irregular ventricular rate, or f waves discharging at a frequency of 350 to 600 beats/min) defined for the atrial fibrillation diagnosis. In response to extracting a first patient indicator—corresponding to the CHA₂DS₂-VASc score recorded for the patient—exceeding the threshold score detecting absence of a second patient indicator—corresponding to the target EKG reading—the computer system can: access a target test method defined for the target EKG reading within the atrial fibrillation module, the target test method configured to yield a patient indicator corresponding to the target EKG reading; and transmit a notification to the provider (e.g., via the provider portal) indicating insufficient evidence included in the provider note for the atrial fibrillation diagnosis and including a suggestion to execute the target test method with the patient. Therefore, the computer system can aid a provider in supporting a diagnosis by suggesting retrieval of additional patient data supporting the diagnosis, in response to the corpus of patient data missing a particular target indicator, thereby reducing costs to patient due to unaccepted provider notes and reducing resources dedicated in generating the note and/or implementing post-hoc corrections due to unaccepted notes.

### 5.9.2 Insufficient Evidence: Multiple Diagnosis Supported

Additionally or alternatively, in another implementation, the computer system can transmit a prompt to the provider suggesting retrieval of additional health data for the patient in response to a set of indicators—extracted from the health record of the patient and supporting a first diagnosis specified by the provider—further supporting a second diagnosis distinct from the first diagnosis. For example, in response to receiving a first diagnosis from a provider, the computer system can: retrieve a first module, contained in the diagnostic model, corresponding to the first diagnosis and defining a first set of target indicators—including a first target indicator, a second target indicator, and a third target indicator—supporting the first diagnosis; scan the health record of the patient for indicators corresponding to the first set of target indicators; and extract a first set of indicators—including a first indicator corresponding to the first target indicator and a second indicator corresponding to the second target indicator—from the health record. The computer system can then present the first and second indicators to the provider within the provider portal. Furthermore, in response to the diagnostic model linking the first module to a second module—corresponding to a second diagnosis and defining a second set of target indicators including the first target indicator, the second target indicator, and a fourth target indicator—the computer system can: retrieve a target test method—defined by the diagnostic model and configured to yield health data corresponding to the third target indicator (e.g., a target blood pressure within a first range) or the fourth target indicator (e.g., a target blood pressure within a second range); generate a notification—indicating insufficient evidence for acceptance of a note specifying the diagnosis—including a suggestion to execute the target test method to further the support the first diagnosis over the second diagnosis; and transmit the notification to the provider within the provider portal.

In one example, during a patient encounter for a patient, the provider can input a pneumonia diagnosis for the patient (e.g., via the provider portal). In particular, in this example, the pneumonia module can define a first set of target indicators including: a first target indicator corresponding to a body temperature exceeding a body temperature threshold; a second target indicator corresponding to a respiratory rate exceeding a respiratory rate threshold; and a third target indicator corresponding to a target chest image (e.g., depicting positive indication of infiltrate, consolidation, density, or opacity within the patient's chest). The computer system can then execute the methods and techniques described above to extract a set of indicators from the patient's health record that support the pneumonia diagnosis. In particular, in this example, the computer system can extract: a first patient indicator—corresponding to a body temperature recorded for the patient—exceeding the body temperature threshold; and a second patient indicator—corresponding to a respiratory rate recorded for the patient—exceeding the respiratory rate threshold. The computer system can then link the pneumonia module to a bronchitis module, via the diagnostic model, corresponding to a bronchitis diagnosis and defining a second set of target indicators. In particular, in this example, the bronchitis module can define the second set of target indicators including the first target indicator, the second target indicator, and a fourth target indicator corresponding to a target chest image (e.g., depicting positive indication of inflammation within a patient's bronchial tubes). In response to detecting the first and second patient indicators supporting the pneumonia and bronchitis diagnoses, the computer system can: access a target test method defined for the third target indicator or the fourth target indicator within the pneumonia module, the target test method configured to yield a patient indicator corresponding to the target chest image supporting the pneumonia or bronchitis diagnoses; and transmit a notification to the provider (e.g., via the provider portal) indicating insufficient evidence included in the provider note for the pneumonia diagnosis and including a suggestion to execute the target test method with the patient to further the support the pneumonia diagnosis over the bronchitis diagnosis. Therefore, the computer system can aid a provider in distinguishing a first diagnosis over a second diagnosis by suggesting retrieval of additional patient data supporting the first diagnosis, in response to the corpus of patient data supporting multiple diagnosis.

### 5.10 Variation: Post-Hoc Note Augmentation

In one variation, the computer system can leverage a set of “hidden” indicators—supporting a diagnosis and initially omitted from a note corresponding to the diagnosis—to update and/or rectify the note responsive to rejection of the note (e.g., by an insurance company). In particular, in this variation, in response to receiving a diagnosis from the provider via the provider portal, the computer system can: implement the methods and techniques described above to present a set of patient indicators—supporting the diagnosis—to the provider for specifying in a note specifying the diagnosis; and, in response to a first selection (e.g., from the provider) of a first subset of patient indicators, in the set of patient indicators, for specifying in the note, append the note with the first subset of patient indicators.

Then, in response to receiving a second selection of a second subset of patient indicators, in the set of patient indicators, for recording in a hidden note (or “secondary note”), the computer system can: withhold appending of the note with the second subset of patient indicators; record the second subset of patient indicators in the hidden note; and store the hidden note—linked to and/or associated with the note—within the health record for the patient. By selecting to record the second subset of patient indicators to the hidden note, the provider may verify these patient indicators as corresponding to the diagnosis, while “hiding” these patient indicators from view and withholding these patient indicators from the note in order to reduce an amount of information—such as information deemed nonessential by the provider—presented within the note and presented to the provider within the provider portal. Later, in response to receiving confirmation of rejection of the note by an entity (e.g., an insurance company) affiliated with the patient, the computer system can automatically: access the hidden note linked to the note; and append the note with the second subset of patient indicators listed in the hidden note to generate a rectified note. Additionally or alternatively, the computer system can: generate a notification including the second subset of indicators and a prompt to select one or more indicators, in the second subset of indicators; and transmit the notification to the provider (e.g., via the provider portal). Then, in response to receiving a third selection of a third subset of patient indicators, in the second subset of patient indicators, from the provider, the computer system can append the rectified note—including the first subset of patient indicators—with the third subset of patient indicators derived from the hidden note and selected by the provider.

In one implementation, the method S100 recites: transmitting a notification to the provider (e.g., via the provider portal) including a prompt to select additional indicators—from the set of patient indicators supporting the diagnosis—for recording in a hidden note in Block S183; recording the subset of patient indicators—selected by the provider—in the hidden note and storing the hidden note, associated with the provider note, within the health record associated with the patient in Block S187; and receiving confirmation of rejection of the provider note in Block S189.

For example, in response to receiving a pneumonia diagnosis for a patient, the computer system can execute methods and techniques described above to: present the provider with a set of three patient indicators—supporting the pneumonia diagnosis—such as including a positive chest image depicting positive indication of infiltrate, a body temperature exceeding a threshold temperature, and a white blood cell count exceeding a threshold count; and prompt the provider to select patient indicators, from the set of three patient indicators, for specifying in a provider note generated for the encounter. n response to receiving selection of a first patient indicator (e.g., the positive chest image)—corresponding to the positive chest image—from the provider (e.g., via the provider portal), for specifying in a provider note(e.g., for distributing to an insurance company and representative of the encounter), the computer system can then generate and/or append the provider note with the first patient indicator. Additionally, in response to receiving selection of a second and third patient indicator—corresponding to the body temperature and the white blood cell count—for specifying in a secondary note (or “hidden note”), the computer system can: generate the secondary note including the second patient indicator corresponding to the body temperature; and link the secondary note—associated with the provider note and/or the encounter—to the health record of the patient for later reference.

Later, in response to receiving confirmation of rejection of the provider note, the computer system can: access the secondary note; generate a notification including the second patient indicator and the third patient indicator—specified in the secondary note—and a prompt to select the second and/or third patient indicator for recording in the (revised) provider note; and, in response to receiving selection of the second patient indicator (e.g., the body temperature) from the provider, append the provider note with the second patient indicator, such that the resulting provider note indicates the pneumonia diagnosis and includes the positive chest image and the body temperature recorded for the patient. Therefore, the computer system can leverage a set of saved and/or “hidden” indicators—supporting a diagnosis and initially omitted from a provider note corresponding to the diagnosis—to update and/or rectify the provider note responsive to rejection of the note (e.g., by an insurance company), thereby reducing resources dedicated, by the provider, to implementing post-hoc corrections due to unaccepted notes.

### 5.11 Augmented Diagnosis

Additionally or alternatively, in one variation, the computer system can present an augmented diagnosis—defining an increased specificity from a provider-supplied diagnosis—to the provider based on the provider-supplied diagnosis and presence of a set of indicators in the patient health record. In particular, in this implementation, the diagnostic model can link supporting evidence and a lower-resolution diagnosis—received from the provider—to a relatively higher-resolution diagnosis. For example, the computer system can: receive an initial diagnosis of “heart failure” from the provider (e.g., via the provider portal); retrieve a first module, in the population of modules stored in the diagnosis model, corresponding to a “heart failure” diagnosis; access a first submodule—linked to the first module—corresponding to a “systolic heart failure” diagnosis and defining a first set of target indicators supporting the “systolic heart failure” diagnosis; and access a second submodule—linked to the first module—corresponding to a “diastolic heart failure” diagnosis and defining a second set of target indicators supporting the “diastolic heart failure” diagnosis. In particular, in this example, the first submodule can define the first set of target indicators including: a first target heart rate within a first heart rate range; and a first target blood pressure within a first blood pressure range. The second submodule can define the second set of target indicators including: a second target heart rate within a second heart rate range; and a second target blood pressure within a second blood pressure range.

In this example, the computer system can then: retrieve the patient's EHR; scan contents of the EHR for indicators corresponding to the first or second set of target indicators; and, in response to the EHR specifying a patient heart rate within the first heart rate range—and therefore corresponding to the first target heart rate—select the “systolic heart failure” diagnosis and suggest the “systolic heart failure” diagnosis to the provider for this patient. For example, the computer system can: flag the “heart failure” diagnosis within the provider portal, such as by highlighting and/or underlining the corresponding text; and render a notification (e.g., a pop-up notification)—suggesting the “systolic heart failure” diagnosis responsive to selection of the highlighted and/or underlined text by the provider. Alternatively, in response to the EHR specifying a patient heart rate within the second range—and therefore corresponding to the second target heart rate—select the “diastolic heart failure” diagnosis and suggest the “diastolic heart failure” diagnosis to the provider within the provider portal.

### 6. New Diagnosis

In one variation, the computer system can selectively suggest a new or “missed” diagnosis—such as an alternative diagnosis to a provider-supplied diagnosis and/or a new or additional diagnosis—based on indicators extracted from a health record of a patient. In particular, in this variation, the computer system can: receive identification of a patient from a provider, such as before, during, or after a patient encounter for this patient; access the diagnostic model containing the population of modules; for a first module, in the population of modules, corresponding to a first diagnosis, identify a first set of target indicators supporting the first diagnosis; scan a health record of the patient for indicators corresponding to the first set of target indicators; extract a set of indicators from the health record corresponding to indicators in the first set of target indicators; and, based on the set of indicators and a set of module parameters defined by the first module, selectively flag and/or withhold flagging of the first diagnosis for further consideration by the provider. The computer system can then repeat this process for each module, in the population of modules, to generate a list of diagnoses corresponding to various health data in the patient's health record; and present the list of diagnoses to the provider within the provider portal for further consideration.

In one implementation, in response to receiving identification of the patient from the provider in Block S115, the computer system can initially: retrieve a set of existing diagnoses, in a population of diagnoses, previously entered by the provider for this patient; and selectively append each diagnosis, in the set of existing diagnoses, with corresponding indicators supporting this diagnosis, as described above. Then, for each other diagnosis (e.g., excluding the set of existing diagnoses), in the population of diagnoses, the computer system can: retrieve a module, in the population of modules, corresponding to the diagnosis; extract a set of target indicators and a set of module parameters—such as defining a set of primary target indicators, a set of secondary target indicators, one or more combinations of target indicators, a minimum quantity of target indicators, etc.—defined for this diagnosis; and selectively flag and/or withhold flagging of the diagnosis for further consideration based on health data contained in the patient's health record, the set of target indicators defined in the module, and the set of module parameters.

### 6.1 Diagnosis Module+Health Record

In one implementation, the computer system can: retrieve a first module, in the population of modules contained in the diagnostic model, corresponding to a first diagnosis; and access a first set of primary target indicators—required to support the first diagnosis—defined by the first module; and scan the health record—including timeseries of health data—for indicators corresponding to the first set of primary target indicators. Then, in response to identifying a first set of indicators—represented in health data contained in the health record—corresponding to the first set of primary indicators, the computer system can: access a first set of secondary target indicators—further supporting the first diagnosis; and scan the health record for indicators corresponding to the first set of secondary target indicators.

Then, in response to identifying a second set of indicators—represented in health data contained in the health record—corresponding to the first set of secondary indicators, the computer system can: generate a suggested diagnosis notification specifying the first diagnosis, the first set of indicators, and/or the second set of indicators; and transmit the suggested diagnosis notification to the provider (e.g., via the provider portal) for review. Alternatively, in response to absence of indicators corresponding to the first set of primary target indicators in the health record, the computer system can: discard the first diagnosis for the patient; retrieve a second module, in the population of modules contained in the diagnostic model, corresponding to a second diagnosis; and repeat this process to selectively flag and/or discard the second diagnosis for the patient.

For example, the computer system can: retrieve a pneumonia module contained in the diagnostic model and corresponding to a pneumonia diagnosis; access a primary target indicator corresponding to a positive chest image—such as a chest image that includes positive indication of infiltrate, consolidation, density, or opacity—defining a target sampling window spanning a patient encounter (e.g., a duration of a hospital visit or clinic); and scan the patient's health record for evidence of a positive chest image captured during the patient encounter. In response to absence of the positive chest image and/or presence of a negative chest image, the computer system can discard the pneumonia diagnosis and retrieve a next module corresponding to a next diagnosis from the diagnostic model. Alternatively, in response to identification of evidence of a positive chest image—captured during the patient encounter—within the health record, the computer system can: access a secondary target indicator corresponding to a target body temperature exceeding a threshold temperature (e.g., 100.4 degrees fahrenheit) and captured for the patient within a target sampling window falling within 48 hours of the positive chest image; and scan the patient's health record for evidence of a body temperature exceeding the threshold temperature and captured within the target sampling window. In response to identifying a body temperature exceeding the threshold temperature and captured for the patient within the target sampling window, the computer system can: flag the first diagnosis as a diagnosis candidate; and/or selectively present the first diagnosis—specifying the primary and secondary target indicators—to the provider (e.g., via the provider portal) for review.

Alternatively, in response to absence of recordation of a body temperature exceeding the threshold temperature within the target sampling window, the computer system can: access a set of tertiary target indicators—including a target WBC exceeding a threshold WBC, a target oxygen saturation falling below a threshold oxygen saturation, and a target respiratory rate exceeding a threshold respiratory rate, each within a target sampling window falling within 24 hours of the positive chest image—supporting the first diagnosis; scan the patient's health record for evidence of the set of tertiary target indicators captured for the patient within the target sampling window; and, based on identification of a quantity of indicators—extracted from the health record and corresponding to the set of tertiary target indicators—exceeding a threshold quantity (e.g., defined by the first module), flag the first diagnosis as a diagnosis candidate and/or selectively present the first diagnosis—specifying the primary target indicator (e.g., the positive chest image) and a subset of tertiary target indicators—to the provider (e.g., via the provider portal) for further review.

Therefore, in this implementation, the computer system can selectively flag and/or discard a particular diagnosis for consideration by the provider based on types (e.g., primary, secondary, tertiary), sampling windows, and/or quantities of target indicators defined by a module corresponding to the particular diagnosis and detection of corresponding indicators in the health record of the patient.

### 6.1.1 Diagnosis Type

Additionally or alternatively, in one implementation, the computer system can supplement a diagnosis suggested to the provider with a particular type, in a set of types, of the diagnosis. For example, the computer system can supplement a pneumonia diagnosis with: a location type including community-acquired, hospital-acquired, or ventilator-acquired pneumonia; and/or a source type including bacterial, viral, a fungal pneumonia. In this example, the computer system can leverage different combinations of target indicators—defined by the module corresponding to pneumonia—to predict a particular type of pneumonia presented by a patient, such as including viral-community-acquired pneumonia, bacterial-ventilator-acquired pneumonia, fungal-hospital-acquired pneumonia, etc.

### 6.1.2 Multiple Modules+List of Diagnoses

Generally, the computer system can access each module in the population of modules and scan the patient health record to generate a list of diagnoses corresponding to various health data in the patient's health record. For example, the computer system can: access a first module corresponding to a first diagnosis; scan a health record associated with a patient for a first set of patient indicators supporting the first diagnosis; selectively approve or reject the first diagnosis for the patient based on the first set of patient indicators; access a second module corresponding to a second diagnosis; scan the health record associated with the patient for a second set of patient indicators supporting the second diagnosis; selectively approve or reject the second diagnosis for the patient based on the second set of patient indicators; access a third module corresponding to a third diagnosis; scan the health record associated with the patient for a third set of patient indicators supporting the third diagnosis; and selectively approve or reject the third diagnosis for the patient based on the third set of patient indicators, etc.

In one implementation, the computer system can leverage the population of modules to: reject a first diagnosis for a patient during a first encounter with the patient; and approve a second diagnosis for the patient during the first encounter. In particular, in this implementation, the computer system can: receive identification of a patient associated with an encounter from the provider (e.g., via the provider portal); for a first module—corresponding to a first diagnosis—access a first set of target indicators supporting the first diagnosis; scan a health record corresponding to the patient for patient indicators corresponding to the first set of target indicators; extract a first subset of patient indicators—from the health record—corresponding to a first subset of target indicators in the first set of target indicators; and derive a first confidence score for the first diagnosis for the patient based on the first subset of patient indicators and the first subset of target indicators. In particular, in this implementation, in response to the first confidence score falling below a threshold score, the computer system can: reject the first diagnosis for the patient for the encounter; access a second module—from the population of modules—corresponding to a second diagnosis and defining a second set of target indicators supporting the second diagnosis; scan the health record for indicators corresponding to the second set of target indicators; extract a second subset of patient indicators—from the health record—corresponding to a second subset of target indicators in the second set of target indicators; and derive a second confidence score for the second diagnosis for the patient based on the second subset of patient indicators and the second subset of target indicators. In response to the second confidence score exceeding the threshold score, the computer system can then: append the list of predicted diagnoses with the second diagnosis; and transmit a notification to the provider (e.g., via the provider portal) including the second subset of patient indicators linked to the second diagnosis.

For example, in response to receiving identification of a patient associated with an encounter from the provider (e.g., via the provider portal), the computer system can: access a pneumonia module corresponding to a pneumonia diagnosis and implement the methods and techniques as described above to derive a first confidence score of 50% for the pneumonia diagnosis for the patient based on a first patient indicator (e.g., a body temperature exceeding a body temperature threshold) supporting the pneumonia diagnosis and extracted from the patient's health record; and, in response to the first confidence score of 50% falling below a threshold score of 75% defined for the pneumonia diagnosis, reject the pneumonia diagnosis for the patient for the encounter. The computer system can then: access a bronchitis module corresponding to a bronchitis diagnosis and implement the methods and techniques as described above to derive a second confidence score of 85% for the bronchitis diagnosis for the patient based on the first patient indicator (e.g., a body temperature exceeding a body temperature threshold) and a second patient indicator (e.g., a target chest image depicting positive indication of inflammation within a patient's bronchial tubes) supporting the bronchitis diagnosis and extracted from the patient's health record; and, in response to the second confidence score exceeding the threshold score, append the list of predicted diagnoses with the bronchitis diagnosis.

Additionally or alternatively, in the preceding example, the computer system can access the bronchitis module defining the bronchitis diagnosis in response to appending the list of predicted diagnoses with the pneumonia diagnosis for the patient. Therefore, the computer system can access each module in the population of modules and scan the patient health record to generate a list of diagnoses corresponding to various health data in the patient's health record. The computer system can repeat this process for each module, in the population of modules, to generate a list of diagnoses corresponding to various health data in the patient's health record; and present the list of diagnoses to the provider within the provider portal for further consideration. The computer system can repeat this process for each module in response to each diagnosis rejected and/or flagged for review.

### 6.2 Deriving a Confidence Score

Block S150 of the method S100 recites deriving a confidence score for the diagnosis for the patient based on the subset of patient indicators extracted from the health record associated with the patient and supporting the diagnosis. In response to identifying patient indicators—represented in the corpus of patient data and corresponding to target indicators supporting the first diagnosis—the computer system can derive a confidence score for the diagnosis for the patient based on the patient indicators and the target indicators.

In one implementation, the computer system can derive the confidence score—representing a confidence in a particular diagnosis for a patient—for each diagnosis in the list of predicted diagnosis. For example, the computer system can predict the confidence score represented by a percentage, a score between 1 and 10, a qualitative score (e.g., low, high, possible, probable), a binary score, etc. The confidence score can be compared to a threshold score—defined in the diagnostic model—to predict a confidence in a particular diagnosis for a patient. In one implementation, the computer system can implement a single threshold score (e.g., a uniform threshold score) for each module in the population of modules. Alternatively, the computer system can implement a variable threshold score defined for each individual module in the population of modules. Generally, in this implementation, the computer system can predict the confidence score for the diagnosis based on the patient indicators present in as patient's health record and supporting the diagnosis. Therefore, the computer system can selectively derive the confidence score for the diagnosis based on identification of patient indicators—from the health record—supporting the diagnosis.

### 6.2.1 Confidence Score: Quantity of Indicators

Additionally, in one implementation, the computer system can supplement a suggested diagnosis presented to the provider with a risk level associated with the diagnosis. For example, the computer system can supplement a diagnosis with a risk level of “negative” diagnosis, “possible” diagnosis, “probable” diagnosis, and/or “positive” diagnosis. In particular, in this implementation, the computer system can: retrieve a first module, in the population of modules contained in the diagnostic model, corresponding to a first diagnosis; access a set of target indicators defined for the first diagnosis; scan the health record—including timeseries of health data—for indicators corresponding to the first set of target indicators; and, selectively predict presence and/or risk of the first diagnosis for the first patient based on identification of indicators—derived from the health record—corresponding to the set of target indicators.

For example, in response to a set of indicators corresponding to four target indicators, in a set of five target indicators defined by the first module, the computer system can characterize risk of the first diagnosis as a “probable” diagnosis. Alternatively, in response to detection of one target indicator, in the set of five target indicators, the computer system can characterize risk of the first diagnosis as a “negative” diagnosis and discard the first diagnosis. In another example, in response to the set of indicators including a first indicator corresponding to a primary target indicator and a second indicator corresponding to a secondary target indicator defined by the first module, the computer system can characterize risk of the first diagnosis as a “positive” diagnosis. Alternatively, in response to the set of indicators omitting an indicator corresponding to the primary target indicator, the computer system can characterize risk of the first diagnosis as a “negative” diagnosis and discard the first diagnosis.

### 6.2.2 Confidence Score: Primary & Secondary Indicators

Additionally or alternatively, in one implementation, the computer system can supplement a suggested diagnosis presented to the provider with a confidence score based on the presence of primary and/or secondary patient indicators supporting a diagnosis. In this implementation, the computer system can: receive, from the provider (e.g., via the provider portal), identification of a patient associated with an encounter; for a module—in the population of modules—corresponding to a diagnosis, extract a primary patient indicator—from the corpus of patient data—corresponding to a primary target indicator required for predicting the diagnosis; and, in response to the primary patient indicator corresponding to the primary target indicator, extract a first secondary patient indicator and a second secondary patient indicator—from the corpus of patient data—corresponding to a first secondary target indicator and a second secondary target indicator supporting the diagnosis in combination with the primary target indicator. In particular, in this implementation, the computer system can: in response to the primary patient indicator corresponding to the primary target indicator, append the list of predicted diagnoses with the first diagnosis labeled as a possible diagnosis; in response to the primary patient indicator corresponding to the primary target indicator, and the first secondary patient indicator corresponding to the first secondary target indicator, append the list of predicted diagnoses with the first diagnosis labeled as a probable diagnosis; in response to the primary patient indicator corresponding to the primary target indicator, the first secondary patient indicator corresponding to the first secondary target indicator, and the second secondary patient indicator corresponding to the second secondary target indicator, append the list of predicted diagnoses with the first diagnosis labeled as a positive diagnosis; and, in response to absence of the primary target indicator in the subset of primary patient indicators, reject the first diagnosis for further investigation.

For example, the computer system can receive identification of a patient associated with an encounter from the provider (e.g., via the provider portal). In particular, in this example, for a pneumonia module corresponding to a pneumonia diagnosis, the computer system can access the pneumonia module defining: a subset of primary target indicators supporting the pneumonia diagnosis and required for predicting the pneumonia diagnosis including a primary target indicator corresponding to a target chest image (e.g., depicting positive indication of infiltrate, consolidation, density, or opacity within the patient's chest); and a subset of secondary target indicators supporting the pneumonia diagnosis in combination with the subset of primary target indicators including a first secondary target indicator corresponding to a white blood cell count (or “WBC”) exceeding a WBC threshold, and a second secondary target indicator corresponding to a respiratory rate exceeding a respiratory rate threshold. For the encounter with the patient, the computer system can: scan a health record—associated with the patient—for the set of primary and secondary target indicators defined for the pneumonia diagnosis; extract a primary patient indicator—corresponding to a positive chest image recorded for the patient depicting positive indication of infiltrate; and, in response to the primary patient indicator corresponding to the primary target indicator, append the list of predicted diagnoses with the pneumonia diagnosis labeled as a possible diagnosis.

Alternatively, in the preceding example, in response to extracting a first secondary patient indicator—corresponding to a WBC recorded for the patient—exceeding the WBC threshold in addition to extracting the primary patient indicator, the computer system can append the list of predicted diagnoses with the pneumonia diagnosis labeled as a probable diagnosis. Alternatively, in the preceding example, in response to extracting a second secondary patient indicator—corresponding to a respiratory rate recorded for the patient—exceeding the respiratory rate threshold in addition to extracting the primary patient indicator and the first secondary patient indicator, the computer system can append the list of predicted diagnoses with the pneumonia diagnosis labeled as a positive diagnosis. Alternatively, in the preceding example, in response to absence of the primary patient indicator—in the health record associated with the patient—the computer system can reject the first diagnosis for further investigation. Therefore, the computer system can derive a confidence score for the diagnosis for the patient based on the presence of primary and secondary patient indicators—from the health record—corresponding to the set of primary and secondary target indicators supporting the diagnosis.

### 6.2.2 Confidence Score: Target Sampling Window

Additionally or alternatively, in one implementation, the computer system can supplement a suggested diagnosis presented to the provider with a confidence score based on the time of recordation of the patient indicator—contained in the corpus of patient data—corresponding to the target indicator. In this implementation, the computer system can: receive identification of a patient associated with an encounter from the provider (e.g., via the provider portal); and, for a module—in the population of modules—corresponding to a diagnosis, access a set of target indicators supporting the diagnosis including a first target indicator defining a first weight assigned to a first target sampling window corresponding to a first time difference between recordation of patient data corresponding to the first target indicator and the encounter, and a second weight assigned to a second target sampling window corresponding to a second time difference between recordation of patient data corresponding to the first target indicator and the encounter. The computer system can then: extract a first patient indicator—from a health record associated with the patient—corresponding to the first target indicator; and, in response to the first patient indicator corresponding to the first target sampling window, derive a confidence score for the diagnosis for the patient based on the first patient indicator, the first target indicator, and the first weight.

Alternatively, in the preceding implementation, in response to the first patient indicator corresponding to the second target sampling window, the computer system can derive the confidence score for the diagnosis for the patient based on the first patient indicator, the first target indicator, and the second weight.

For example, the computer system can receive identification of a patient associated with an encounter from the provider (e.g., via the provider portal). For a pneumonia module—in the population of modules—corresponding to a pneumonia diagnosis, the computer system can access a set of target indicators supporting the pneumonia diagnosis including a first target indicator of a target chest image defining: a first weight assigned to a first target sampling window corresponding to a first time difference between recordation of patient data corresponding to the first target indicator and the encounter (e.g., 24 hours); and a second weight, less than the first weight, assigned to a second target sampling window corresponding to a second time difference between recordation of patient data corresponding to the first target indicator and the encounter (e.g., 72 hours). For the encounter with the patient, the computer system can: extract a first patient indicator—corresponding to a positive chest image recorded for the patient within 24 hours of the encounter—depicting positive indication of infiltrate; and derive a confidence score for the pneumonia diagnosis for the patient based on the first patient indicator, the first target indicator, and the first weight. Therefore, the computer system can derive a confidence score for the diagnosis for the patient based on a particular weight assigned to a patient indicator based on the time of recordation—contained in the corpus of patient data and supporting the diagnosis—corresponding to the target indicator.

### 6.2.4 Confidence Score: Target Indicator Weights

Additionally or alternatively, in one implementation, the computer system can supplement a suggested diagnosis presented to the provider with a confidence score based on a patient indicator—contained in the corpus of patient data—corresponding to the target indicator assigned a particular weight. In this implementation, the computer system can: receive identification of a patient associated with an encounter from the provider (e.g., via the provider portal); and, for a module—in the population of modules—corresponding to a diagnosis, access a set of target indicators supporting the diagnosis including a first target indicator assigned a first weight, and a second target indicator assigned a second weight less than the first weight. For the encounter with the patient, the computer system can extract a first patient indicator—from the corpus of patient data—corresponding to the first target indicator; and derive a confidence score for the diagnosis for the patient based on the first patient indicator, the first target indicator, and the first weight.

For example, the computer system can: receive identification of a patient associated with an encounter from the provider (e.g., via the provider portal); and, for a pneumonia module—in the population of modules—corresponding to a pneumonia diagnosis, access a set of target indicators supporting the pneumonia diagnosis including a first target indicator of a target chest image (e.g., depicting positive indication of infiltrate, consolidation, density, or opacity within the patient's chest) assigned a first weight, and a second target indicator corresponding to a respiratory rate exceeding a respiratory rate threshold—assigned a second weight less than the first weight. For the encounter with the patient, the computer system can: scan a health record—associated with the patient—for the set of target indicators defined for the pneumonia diagnosis; extract a first patient indicator—corresponding to a positive chest image recorded for the patient—depicting positive indication of infiltrate; and derive a confidence score of 75% for the pneumonia diagnosis for the patient based on the first patient indicator, the first target indicator, and the first weight.

Alternatively, in the preceding example, in response to absence of the first patient indicator and presence of a second patient indicator—corresponding to a respiratory rate recorded for the patient—exceeding the respiratory rate threshold, the computer system can derive a confidence score of 25% for the pneumonia diagnosis for the patient based on the second patient indicator, the second target indicator, and the second weight.

### 6.3 Presenting the Diagnosis

Block S167 of the method S100 recites in response to the confidence score exceeding a threshold score: appending a list of predicted diagnoses with the diagnosis; generating a notification including the list of predicted diagnoses and a prompt to review the list of predicted diagnoses; populating the notification with the subset of patient indicators linked to the diagnosis; and, via the provider portal, transmitting the notification to the provider for review. In response to flagging a diagnosis for further investigation by the provider, the computer system can selectively present the diagnosis to the provider via the provider portal. In particular, in response to flagging the diagnosis for further investigation, the computer system can: pair the diagnosis with a set of indicators extracted from the patient's health record and supporting the diagnosis; and present the diagnosis and the set of indicators within the provider portal, such as below a set of existing diagnoses previously entered and/or selected by the provider.

In one implementation, the computer system can selectively order the list of diagnoses according to confidence and/or “risk” associated with each diagnosis in the list. For example, the computer system can: present a first diagnosis—defining a first confidence score—in a first slot in the list of diagnoses; present a second diagnosis—defining a second confidence score falling below the first confidence score—in a second slot below the first slot in the list of diagnoses; and present a third diagnosis—defining a third confidence score falling below the second confidence score—in a third slot below the second slot in the list of diagnoses. Additionally or alternatively, in another implementation, the computer system can selectively order the list of diagnoses according to urgency or patient risk. For example, the computer system can: present a first diagnosis of “appendicitis”—defining a relatively high urgency level—in a first slot in the list of diagnoses; and present a second diagnosis of “arthritis” in a second slot below the first slot in the list of diagnoses.

### 6.3.1 Variation: Presenting the Diagnosis+Confidence Score

In response to generating a list of predicted diagnoses, the computer system can selectively present the predicted diagnosis to the provider within the provider portal. In one implementation, the computer system can selectively present the diagnosis based on the confidence score derived for each diagnosis. In this implementation, for a first module—in the population of modules—corresponding to a first diagnosis, the computer system can: execute the methods and techniques described above to extract a first set of patient indicators from the patient's health record that support the first diagnosis; derive a first confidence score for the first diagnosis for the patient; and, in response to the first confidence score exceeding the threshold score, append the list of predicted diagnoses with the first diagnosis. Then, for a second module—in the population of modules—corresponding to a second diagnosis, the computer system can: execute the methods and techniques described above to extract a second set of patient indicators from the patient's health record that support the second diagnosis; derive a second confidence score for the second diagnosis for the patient; and, in response to the second confidence score exceeding the threshold score, append the list of predicted diagnoses with the second diagnosis. In particular, in this implementation, in response to the first confidence score exceeding the second confidence score, the computer system can: append the list of predicted diagnoses with the first diagnosis in a first slot in the list of predicted diagnoses; and append the list of predicted diagnoses with the second diagnosis in a second slot, below the first slot, in the list of predicted diagnoses.

For example, the computer system can: receive identification of a patient associated with an encounter from the provider (e.g., via the provider portal); access a pneumonia module—in the population of modules contained in the diagnostic model—corresponding to the pneumonia diagnosis; extract a first patient indicator—from a health record associated with the patient—corresponding to a first target indicator supporting the pneumonia diagnosis; and derive a first confidence score of 60% for the pneumonia diagnosis for the patient. The computer system can then: access a bronchitis module—from the population of modules—corresponding to a bronchitis diagnosis; extract a second patient indicator—from the health record—corresponding to a second target indicator supporting the bronchitis diagnosis; extract a third patient indicator—from the health record—corresponding to a third target indicator supporting the bronchitis diagnosis; and derive a second confidence score of 80% for the bronchitis diagnosis for the patient based. In particular, in this example, in response to the second confidence score exceeding the first confidence score, the computer system can: append the list of predicted diagnoses with the bronchitis diagnosis in a first slot in the list of predicted diagnoses; and append the list of predicted diagnoses with the pneumonia diagnosis in a second slot, below the first slot, in the list of predicted diagnoses. Therefore, the computer system can selectively present the predicted diagnoses by ordering the diagnoses within the list based on confidence score, thereby promoting review of a particular diagnosis having a greater confidence score and limiting presentation of lower confidence diagnosis to the provider.

### 6.3.2 Variation: Presenting the Diagnosis+Urgency Level

Additionally or alternatively, in one implementation, the computer system can selectively present the diagnosis based on an urgency level—defined by the diagnostic model—for each diagnosis, the urgency level representing the risk to the patient presented by a particular diagnosis. In particular, in this implementation, for a first module corresponding to a first diagnosis, the computer system can execute the methods and techniques described above to: predict the first diagnosis for the patient; and, for a second module corresponding to a second diagnosis, predict the second diagnosis for the patient. Then, in response to a first urgency level assigned to the first diagnosis exceeding a second urgency level assigned to the second diagnosis, the computer system can: append the list of predicted diagnoses with the first diagnosis in a first slot in the list of predicted diagnoses; and append the list of predicted diagnoses with the second diagnosis in a second slot, below the first slot, in the list of predicted diagnoses. In one example, the computer system can: access a heart failure module corresponding to a heart failure diagnosis and execute the methods and techniques described above to predict the heart failure diagnosis for the patient; and, for an arthritis module corresponding to an arthritis diagnosis, execute the methods and techniques described above to predict the arthritis diagnosis for the patient. Then, in response to a first urgency level assigned to the heart failure diagnosis exceeding a second urgency level assigned to the arthritis diagnosis, the computer system can present the heart failure diagnosis in a first slot near a top of the list of predicted diagnoses. Therefore, the computer system can selectively present the predicted diagnoses by ordering the diagnoses within the list based on the urgency level defined for a particular diagnosis, thereby promoting review of a particular diagnosis presenting a greater risk to the patient and limiting presentation of lower risk diagnoses to the provider.

### 6.3.3 Variation: Subdiagnosis

Additionally or alternatively, in one implementation, the computer system can selectively present the diagnosis based on the presence of indicators supporting a subdiagnosis. In this implementation, the computer system can implement a diagnostic model including a population of modules and a population of submodules—corresponding to specific types and/or subtypes of generic diagnoses represented in the population of modules—linked to and/or integrated within the population of modules. For example, the computer system can execute the methods and techniques described above to predict heart failure diagnosis for a patient. The computer system can then access the heart failure module including: a first submodule—corresponding to a systolic heart failure diagnosis as the first subdiagnosis of the heart failure diagnosis—including a first subset of target indicators including a first target heart range and a first target blood pressure range, the first subset of target indicators supporting the systolic heart failure diagnosis; and a second submodule—corresponding to a diastolic heart failure diagnosis as the second subdiagnosis of the heart failure diagnosis—including a second subset of target indicators including a second target heart rate and a second target blood pressure, the second subset of target indicators supporting the diastolic heart failure diagnosis. In this example, the computer system can then: retrieve the patient's EHR; scan contents of the EHR for indicators corresponding to the first or second set of target indicators; and, in response to the EHR specifying a patient heart rate within the first heart rate range—and therefore corresponding to the first target heart rate—select the “systolic heart failure” diagnosis and suggest the “systolic heart failure” diagnosis to the provider for this patient. For example, the computer system can: flag the “heart failure” diagnosis within the provider portal, such as by highlighting and/or underlining the corresponding text; and render a notification (e.g., a pop-up notification)—suggesting the “systolic heart failure” diagnosis responsive to selection of the highlighted and/or underlined text by the provider. Alternatively, in response to the EHR specifying a patient heart rate within the second range—and therefore corresponding to the second target heart rate—select the “diastolic heart failure” diagnosis and suggest the “diastolic heart failure” diagnosis to the provider within the provider portal. Therefore, the computer system can selectively present a predicted subdiagnosis defining an increased specificity from a particular diagnosis based on presence of a set of indicators in the patient health record.

### 6.4 Generating the Provider Note

Generally, as described above, the computer system can host or interface with the provider portal—accessed by the provider—to aid the provider in producing a provider note (e.g., an electronic progress note, an electronic clinical note) containing relevant information for a corresponding encounter (or “patient encounter”) between a patient and the provider. In one implementation, the computer system can host or interface with the provider portal to generate a provider note indicating a particular diagnosis—suggested to the provider—in response to selection of the particular diagnosis in a list of predicted diagnosis transmitted to the provider. For example, in this implementation, the computer system can: initialize a provider note for a first encounter with a patient; implement methods and techniques described above to generate and transmit a list of predicted diagnoses to the provider; and, in response to receiving acceptance of a diagnosis, in the list of predicted diagnoses, by the provider, append the provider note with the diagnosis. The computer system can additionally append the provider note with: a set of patient indicators supporting the diagnosis; and/or a treatment pathway, in a set of treatment pathways, selected by the provider (e.g., via the provider portal) and predicted to treat the diagnosis.

### 6.4.1 Variation: Diagnosis Record

Block S190 of the method S100 recites: generating a diagnosis record for the first encounter with the patient; and, in response to receiving acceptance of a diagnosis by the provider, storing a first data packet with a first value indicating acceptance of the first diagnosis by the provider, in a series of diagnosis records associated with the patient, within a record database. Additionally or alternatively, in one variation, the computer system can generate and maintain a diagnosis record of historical diagnoses for the patient, such as including all diagnoses predicted for the patient, diagnoses accepted by the provider, diagnoses rejected by the provider, etc.

In particular, in one implementation, the computer system can: implement Blocks of the method S100, as described above, to suggest a particular diagnosis to the provider via the provider portal; generate a diagnosis record for the encounter with a patient; populate a data packet with the particular diagnosis and a subset of patient indicators—extracted from the health record of the patient—supporting the diagnosis; and store the data packet in the diagnosis record.

Additionally or alternatively, in the preceding implementation, in response to receiving acceptance of the diagnosis by the provider, the computer system can: populate the data packet with a first value indicating acceptance of the diagnosis; and store the diagnosis record, in a series of diagnosis records associated with the patient, within a record database. For example, the computer system can: implement Blocks of the method S100, as described above, to suggest an anemia diagnosis to the provider (e.g., via the provider portal) for a patient; generate a diagnosis record for the encounter with the patient; and generate a data packet including the anemia diagnosis and the subset of patient indicators supporting the anemia diagnosis (e.g., a hemoglobin level recorded for the patient—falling below a hemoglobin threshold and a hematocrit level recorded for the patient—falling below a hematocrit threshold). Then, in response to receiving acceptance of the anemia diagnosis by the provider, the computer system can: populate the data packet with a first value indicating acceptance of the anemia diagnosis; and store the diagnosis record, in a series of diagnosis records associated with the patient, within a record database. Alternatively, in response to receiving rejection of the anemia diagnosis by the provider, the computer system can: populate the data packet with a second value indicating rejection of the anemia diagnosis by the provider; and store the anemia diagnosis as a possible diagnosis within the diagnosis record. Therefore, the computer system can: store historical diagnoses for a particular patient within a diagnosis record; and access the diagnosis record in successive patient encounters, thereby improving documentation of historical patient data.

### 6.5 Insufficient Evidence

In one variation, the computer system can suggest retrieval of additional patient data to investigate a possible diagnosis, such as in response to the corpus of patient data missing a primary patient indicator required for supporting a diagnosis. For example, the computer system can suggest the retrieval of patient vitals, lab results, imaging results, etc. In this variation, the computer system can: receive identification of a patient associated with an encounter from the provider (e.g., via the provider portal); and, for a module—in the population of modules—corresponding to a diagnosis, access a set of target indicators supporting the diagnosis including a subset of primary target indicators including a primary target indicator required for predicting the diagnosis, and a subset of secondary target indicators including a first secondary target indicator and a second secondary target indicator supporting the diagnosis in combination with the set of primary target indicators. Then, in response to extracting a subset of secondary patient indicators—from the corpus of patient data—corresponding to each secondary target indicator in the subset of secondary target indicators, the computer system can: detect absence of a primary patient indicator—in the corpus of patient data—corresponding to the primary target indicator; access a target test method defined for the primary target indicator within the module, the target test method configured to yield a patient indicator corresponding to the primary target indicator; and transmit a notification to the provider (e.g., via the provider portal) indicating insufficient evidence for the diagnosis and including a suggestion to execute the target test method with the patient.

For example, the computer system can receive identification of a patient associated with an encounter from the provider (e.g., via the provider portal). For an atrial fibrillation module—in the population of modules—corresponding to an atrial fibrillation diagnosis, the computer system can access: a subset of primary target indicators supporting the atrial fibrillation diagnosis and required for predicting the atrial fibrillation diagnosis including a primary target indicator corresponding to a CHA₂DS₂-VASc score exceeding a threshold score and derived from patient health data including instances of prior stroke(s), patient diagnoses of diabetes, hypertension, heart failure, and/or vascular disease, patient age, patient sex, etc.; and a subset of secondary target indicators supporting the atrial fibrillation diagnosis in combination with the subset of primary target indicators including a first secondary target indicator corresponding to a heart rate within a target heart rate range, and a second secondary target indicator corresponding to a respiratory rate exceeding a respiratory rate threshold.

For the encounter with the patient, the computer system can: scan a health record—associated with the patient—for the subset of primary and secondary target indicators defined for the atrial fibrillation diagnosis; extract a first secondary patient indicator—corresponding to a heart rate recorded for the patient—falling within the target heart rate range; and extract a second secondary patient indicator—corresponding to a respiratory rate recorded for the patient—exceeding the respiratory rate threshold. In response to detecting absence of a CHA₂DS₂-VASc score, in the corpus of patient data, corresponding to the target CHA₂DS₂-VASc score, the computer system can: access a target test method defined for the CHA₂DS₂-VASc score within the atrial fibrillation module, the target test method configured to yield a CHA₂DS₂-VASc score corresponding to the target CHA₂DS₂-VASc score; and transmit a notification to the provider (e.g., via the provider portal) indicating insufficient evidence for the atrial fibrillation diagnosis and including a suggestion to execute the target test method with the patient. Therefore, the computer system can suggest retrieval of additional health data for a patient in order to further investigate a possible diagnosis, such as in response to detecting absence of a primary patient indicator required for supporting a diagnosis.

In one variation, the computer system can suggest retrieval of additional health data for a patient in order to further investigate a possible diagnosis, such as in response to confidence in a positive diagnosis falling below a threshold confidence. For example, the computer system can: retrieve a first module—corresponding to a first diagnosis—defining a primary indicator (e.g., abnormal WBC) and a set of secondary indicators (e.g., high blood pressure, elevated resting heart rate, nausea) supporting the first diagnosis; and scan the patient's EHR for indicators corresponding to the primary indicator and the set of secondary indicators. Then, in response to extracting a first set of indicators corresponding to each secondary indicator, in the set of secondary indicators, the computer system can: flag the first diagnosis as a possible diagnosis; generate a notification indicating the possible diagnosis and including a suggestion to order a particular patient exam—defined in the first module—corresponding to the primary indicator; and transmit the notification to the provider within the provider portal. Later, in response to receiving new patient data for this patient, the computer system can: scan this new patient data; and, in response to extracting a first indicator corresponding to the primary indicator, flag the first diagnosis for further investigation by the provider.

In one example, the computer system can receive identification of a patient associated with an encounter from the provider (e.g., via the provider portal). In particular, in this example, the computer system can access an atrial fibrillation module defining a set of four target indicators including: a first target indicator corresponding to a CHA₂DS₂-VASc score exceeding a threshold score and derived from patient health data including instances of prior stroke(s), patient diagnoses of diabetes, hypertension, heart failure, and/or vascular disease, patient age, patient sex, etc.; a second target indicator corresponding to a target electrocardiogram (or “EKG”) reading (e.g., depicting absence of P-waves, an irregular ventricular rate, or f waves discharging at a frequency of 350 to 600 beats/min); a third target indicator corresponding to a heart rate within a target heart rate range; and a fourth target indicator corresponding to a respiratory rate exceeding a respiratory rate threshold. For the encounter with the patient, the computer system can: scan a health record—associated with the patient—for the set of four target indicators defined for the atrial fibrillation diagnosis; extract a first patient indicator—corresponding to the CHA₂DS₂-VASc score recorded for the patient—exceeding the threshold score; extract a second patient indicator—corresponding to a heart rate recorded for the patient—falling within the target heart rate range; and derive a confidence score of 50% for the atrial fibrillation diagnosis for the patient. In particular, in this example, in response to the confidence score falling below the threshold score of 75% and exceeding a minimum threshold score of 40%, defined for the atrial fibrillation diagnosis, the computer system can: access a target test method defined for the second target indicator, defined within the atrial fibrillation module, the target test method configured to yield a patient indicator corresponding to the target EKG reading; and transmit a notification to the provider (e.g., via the provider portal) indicating insufficient evidence for the atrial fibrillation diagnosis and including a suggestion to execute the target test method with the patient. Therefore, the computer system can suggest retrieval of additional health data for the patient in order to further investigate a possible diagnosis, such as in response to the confidence score falling below a threshold score and exceeding a minimum threshold score.

Additionally or alternatively, in this variation, the computer system can suggest retrieval of additional health data for a patient in order to distinguish between multiple diagnoses defining similar and/or overlapping target indicators. For example, the computer system can: retrieve a first module corresponding to a first diagnosis and defining a first target indicator, a second target indicator, and a third target indicator; extract a first and second indicator—corresponding to the first and second target indicators—from the patient's health record; and flag the first diagnosis for further investigation. The computer system can further: retrieve a second module corresponding to a second diagnosis and defining the first target indicator, the second target indicator, and a fourth target indicator; extract the first and second indicator—corresponding to the first and second target indicators—from the patient's health record; and flag the second diagnosis for further investigation. Then, in response to flagging both the first and second diagnoses—based on the first and second target indicators for each diagnosis—the computer system can: identify a patient exam corresponding to both the third target indicator (e.g., a low blood pressure) and the fourth target indicator (e.g., a high blood pressure), such that execution of the patient exam may yield results corresponding to either the third target indicator or the fourth target indicator; generate a notification indicating the possible first and second diagnoses and including a suggestion to execute the patient exam; and transmit the notification to the provider via the provider portal. Later, in response to receiving new patient data for this patient, the computer system can: scan this new patient data; and, in response to extracting a third indicator corresponding to the third target indicator, flag the first diagnosis for further investigation by the provider and discard the second diagnosis.

### 6.6 Updating the Diagnosis

In one implementation, the computer system can update a particular diagnosis over time as new patient data is collected for the patient. In particular, in this implementation, the computer system can: at a first time, implement methods and techniques described above to flag a diagnosis and present this diagnosis to the provider (e.g., within the provider portal) for review and/or further investigation; at a second time, access a set of new health data—including vitals, labs, images, etc.—captured for the patient during a time period succeeding the first time and preceding the second time; access a set of target indicators defined for the diagnosis in a module contained in the diagnostic model; scan the set of new health data for indicators corresponding to the set of target indicators; and selectively update presentation of the diagnosis based on presence of indicators in the new health data corresponding to and/or conflicting with the set of target indicators.

In one example, in response to extraction of an additional or new indicator—corresponding to a target indicator in the set of target indicators—from the new health data, the computer system can: append the list of indicators presented with the diagnosis to include the new indicator; update a risk assigned to the diagnosis from “possible” to “probable” or “positive”; update a type of the diagnosis based on the new indicator; and/or direct the provider's attention to this suggested diagnosis, such as by surfacing the diagnosis at a top of a list of suggested diagnoses, rendering a notification indicating a diagnosis update, highlighting the diagnosis and/or the new indicator, etc. Alternatively, in response to extraction of an indicator conflicting with the diagnosis, the computer system can automatically discard the diagnosis or prompt the provider to review the diagnosis and/or verify discarding of the diagnosis.

In another example, the computer system can execute the methods and techniques described above to derive a confidence score of 50% for an atrial fibrillation diagnosis for the patient based on a first patient indicator (e.g., corresponding to the CHA₂DS₂-VASc score recorded for the patient—exceeding the threshold score) and a second patient indicator (e.g., corresponding to a heart rate recorded for the patient—falling within the target heart rate range) extracted from the patient's health record. In particular, in this example, in response to the confidence score of 50% falling below the threshold score of 75% and exceeding a minimum threshold score of 40%, defined for the atrial fibrillation diagnosis, the computer system can store the atrial fibrillation diagnosis as a possible diagnosis within a diagnosis record generated for the first encounter with the patient. For a second encounter with the patient, the computer system can: extract a third patient indicator (e.g., corresponding to an EKG reading—captured for the patient during a time period succeeding the first encounter and preceding the second encounter—corresponding to the target EKG reading); and derive a second confidence score of 100% for the atrial fibrillation diagnosis for the patient based on the first patient indicator, the second patient indicator, and the third patient indicator. The computer system can then execute the methods and techniques described above to present the atrial fibrillation diagnosis to the provider (e.g., via the provider portal).

Alternatively, in the preceding example, in response to a third subset of patient indicators—in the new set of patient indicators—conflicting with the set of target indicators and the atrial fibrillation diagnosis, the computer system can append the diagnosis record with a list of rejected diagnoses including the atrial fibrillation diagnosis. Therefore, the computer system can update a particular diagnosis over time as new patient data is collected for the patient, thereby reducing resources dedicated by the provider in successive encounters with a patient.

### 6.7 Variation: Critical Value Notification

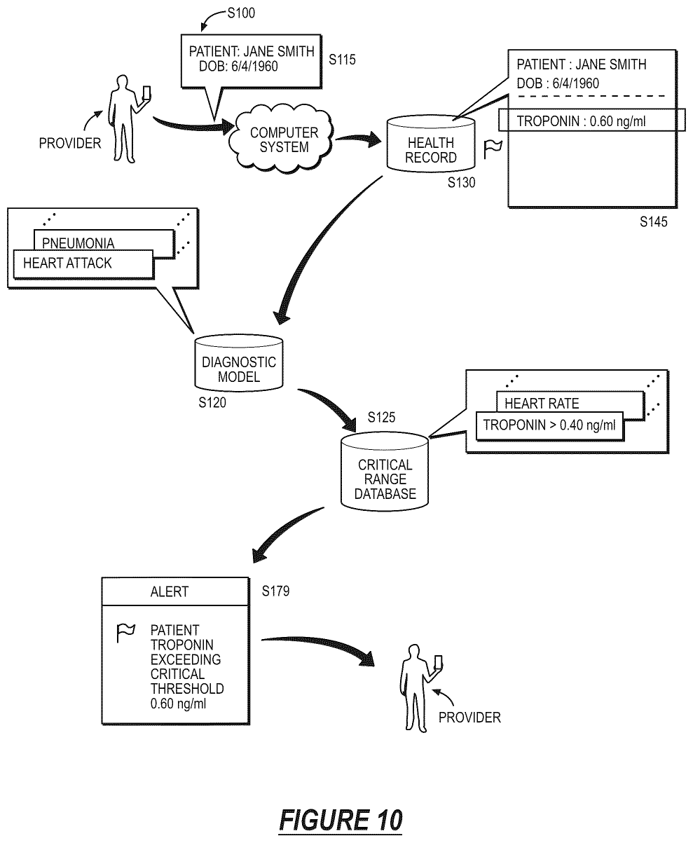

As shown in FIG. 10, in one variation, Blocks of the method S100 can include: accessing a critical range defined for each indicator, in a population of indicators, from a critical range database in Block S125; extracting a first patient indicator, from the patient health record, corresponding to an indicator in Block S145; and, in response to the first patient indicator falling within a critical range defined for the indicator, alerting the provider via the provider portal of the first patient indicator and the critical range defined for the indicator in Block S179. In this variation, the computer system can alert the provider of patient indicators—in the corpus of patient data—that fall within critical ranges defined for each indicator within a critical range database, the critical range database defining a high-risk range for each indicator.

In one implementation, the computer system can: receive new patient data—in a health record associated with a patient—including a set of new patient indicators such as, in response to the provider executing additional test methods with the patient for an encounter; extract a first patient indicator—from the corpus of patient data—corresponding to a first indicator; and access a critical range defined for the indicator in the critical range database. Then, in response to the first patient indicator falling within a first critical range defined for the first indicator, the computer system can alert the provider (e.g., via the provider portal) of the first patient indicator and the first critical range defined for the first indicator. For example, the computer system can: extract a troponin reading recorded for the patient of 0.60 ng/ml; access a troponin critical threshold specifying a troponin level exceeding 0.40 ng/ml as high-risk to the patient; and alert the provider (e.g., via the provider portal) of the patient troponin reading and the troponin critical range defined for the troponin indicator. Therefore, the computer system can alert the provider of patient indicators—in the corpus of patient data—that fall within critical ranges defined for each indicator within a critical range database, thereby alerting the provider of indicators that require immediate attention.

### 7. Variation: Whitelist & Blacklist Medications

In one variation, as shown in FIGS. 3 and 9, the computer system can implement a diagnostic model linking each diagnosis, in a population of diagnoses, to a set of whitelisted medications associated with treatment and/or management of the diagnosis and/or to a set of blacklisted medications predicted to exacerbate the diagnosis.

In particular, in this variation, for a particular diagnosis, the computer system can: populate a medication whitelist with a set of “whitelisted” medications configured to elicit a target response (e.g., a positive response) responsive to implementation by a patient diagnosed with the particular diagnosis; populate a medication blacklist with a set of “blacklisted” medications—such as including one or more “blacklisted” compounds or ingredients—configured to aggravate and/or exacerbate the particular diagnosis for the patient; and store the medication whitelist and the medication blacklist in a particular module—corresponding to the particular diagnosis—in the diagnostic model. Then, in response to a new diagnosis for a patient—such as entered by the provider within the provider portal and/or predicted by the computer system based on target indicators derived from a timeseries of health data associated with the patient—corresponding to the particular diagnosis, the computer system can: access a current set of medications implemented by the patient and represented in the patient's health record; scan the current set of medications for medications included in the medication whitelist; and scan the current set of medications for medications included in the medication blacklist. Then, in response to the medication whitelist including a first medication, in the current set of medications, the computer system can: verify implementation of the first medication for the patient; and populate a medication report with the first medication and indicate verification of the first medication for the patient and/or for the particular diagnosis within the medication report. Furthermore, in response to the medication blacklist including a second medication, in the current set of medications, the computer system can: flag the second medication for further review by the provider; generate a notification indicating implementation of the second medication by the patient and inclusion of the second medication in the medication blacklist for this particular diagnosis; populate the medication report with the notification; and present the medication report to the provider within the provider portal.

Additionally, the computer system can highlight the notification in order to draw the provider's attention to this notification and prompt review of the second medication for this patient. For example, the computer system can: populate the medication report with the notification at a top of the medication report; visually emphasizing text of the notification (e.g., emboldened, highlighted, and/or color-coded text); alert the provider to review the notification within the provider portal, such as by rendering a “pop-up” alert within the provider portal and/or animating the notification; etc.

The computer system can therefore: receive a new diagnosis for a patient from a provider and/or automatically predict a new diagnosis for the patient; access whitelisted and blacklisted medications defined for this new diagnosis in a module—corresponding to the new diagnosis—of the diagnostic module; scan a list of current medications implemented by the patient for medications whitelisted and blacklisted medications defined in the module; and, in response to the list of current medications including one or more blacklisted medications defined for the new diagnosis, immediately notify the provider to review the one or more blacklisted medications—implemented by the patient—in order to minimize exacerbation of the new diagnosis and/or worsening of the patient's health. Additionally, in response to the list of current medications including one or more whitelisted medications defined for the new diagnosis, the computer system can notify the provider and/or populate a note—generated for the patient encounter—with the whitelisted medication accordingly.

In this variation, Blocks of the method S100 can include, in response to receiving confirmation of a patient encounter—for a patient—from a provider via an instance of the provider portal: retrieving a diagnostic model containing a population of modules, each module, in the population of modules, defining a particular diagnosis in a population of diagnoses in Block S120; for a first module, in the population of modules, defining a first diagnosis, extracting a set of target indicators required for supporting the first diagnosis in Block S122; accessing an electronic health record—including a timeseries of health data—associated with the patient in Block S130; and, in response to the timeseries of health data defining a first set of indicators corresponding to the set of target indicators, appending a list of possible diagnoses with the first diagnosis. In this variation, the method S100 further includes, in response to appending the list of possible diagnoses with the first diagnosis: extracting a list of current medications implemented by the patient from the timeseries of health data in Block S135; and accessing a medication blacklist—including a set of blacklisted medications configured to exacerbate the first diagnosis—defined for the first diagnosis in the first module in Block S128. In response to the list of current medications including a first medication, in the set of blacklisted medications, the method S100 further includes: flagging the first medication for further review by the provider in Block S168; and generating a notification—indicating implementation of the first medication by the patient and inclusion of the first medication in the medication blacklist defined for the first diagnosis—including a prompt to review the first diagnosis and implementation of the first medication by the patient, populating a report with the list of possible diagnoses and the notification, and transmitting the report to the provider—via the provider portal—for review in Block S167.

Additionally, in one variation, the method S100 can further include, in response to appending the list of possible diagnoses with the first diagnosis: accessing a medication whitelist—including a set of whitelisted medications configured to treat the first diagnosis—defined for the first diagnosis in the first module in Block S127. In response to the list of current medications including a second medication, in the set of whitelisted medications, the method S100 further includes: flagging the second medication as an approved medication for treating the first diagnosis in Block S169; and generating a second notification—indicating implementation of the second medication by the patient and inclusion of the second medication in the medication whitelist defined for the first diagnosis—including a second prompt to review implementation of the second medication by the patient (e.g., for treatment of the first diagnosis), populating the report with the list of possible diagnoses and the second notification and transmitting the report to the provider—via the provider portal—for review in Block S167.

### 7.1 New Diagnosis

In one implementation, the computer system can receive a new diagnosis entered by a provider for the patient during an encounter, such as via the provider portal executing on a computing device (e.g., a tablet, a desktop computer, a smartphone) accessed by the provider. In response to receiving the diagnosis, the computer system can automatically: access a medication whitelist defined for the diagnosis; access a medication blacklist defined for the diagnosis; access a list of current medications implemented by the patient; identify one or more medications—currently implemented by the patient—included in the medication whitelist and/or blacklist; and selectively notify the provider (e.g., via the provider portal) of these one or more medications accordingly, such as in near real-time.

Additionally or alternatively, in one implementation, as described above, the computer system can selectively suggest a new or “missed” diagnosis—such as an alternative diagnosis to a provider-supplied diagnosis and/or a new or additional diagnosis—based on indicators extracted from a health record of a patient. In particular, in this variation, the computer system can: receive identification of a patient from a provider, such as before, during, or after a patient encounter for this patient; access the diagnostic model containing the population of modules; for a first module, in the population of modules, corresponding to a first diagnosis, identify a first set of target indicators supporting the first diagnosis; scan a health record of the patient for indicators corresponding to the first set of target indicators; extract a set of indicators from the health record corresponding to indicators in the first set of target indicators; and, based on the set of indicators and a set of module parameters defined by the first module, selectively flag and/or withhold flagging of the first diagnosis for further consideration by the provider. The computer system can then repeat this process for each module, in the population of modules, to generate a list of diagnoses corresponding to various health data in the patient's health record; and present the list of diagnoses to the provider within the provider portal for further consideration.

Then, in response to presenting a particular diagnosis to the provider for review, the computer system can automatically: access a medication whitelist defined for the particular diagnosis; access a medication blacklist defined for the particular diagnosis; access a list of current medications implemented by the patient; identify one or more medications—currently implemented by the patient—included in the medication whitelist and/or blacklist; and selectively notify the provider (e.g., via the provider portal) of these one or more medications accordingly, such as in near real-time. Therefore, in this implementation, the computer system can rapidly notify the provider of possible missed diagnoses for the patient and medications—employed by the patient—that may exacerbate these possible missed diagnoses.

### 7.2 Diagnosis Model

Generally, the computer system can implement a diagnostic model linking each diagnosis, in a population of diagnoses, to: a set of supporting evidence (or “indicators”)—such as including units and/or combinations of evidence (e.g., vitals, lab results, imaging results)—that support the diagnosis; a medication whitelist including a set of whitelisted medications configured to treat the diagnosis; and/or a medication blacklist including a set of blacklisted medications configured to exacerbate the diagnosis. For example, the computer system can implement a diagnostic model linking: a first diagnosis to a first set of target indicators (e.g., derived from patient vitals, labs, images), a first set of whitelisted medications configured to treat the first diagnosis, and a first set of blacklisted medications configured to exacerbate the first diagnosis; a second diagnosis to a second set of target indicators, a second set of whitelisted medications configured to treat the second diagnosis, and a second set of blacklisted medications configured to exacerbate the second diagnosis; a third diagnosis to a third set of target indicators, a third set of whitelisted medications configured to treat the third diagnosis, and a third set of blacklisted medications configured to exacerbate the third diagnosis; etc.

In one implementation, the diagnostic model can include a population of modules, each module, in the population of modules, corresponding to a particular diagnosis in a population of diagnoses. In particular, each module (e.g., a data container), in the population of modules, can define: a diagnosis; a set of target indicators associated with (e.g., supporting) the diagnosis; a set of whitelisted medications configured to treat the diagnosis; and a set of blacklisted medications configured to exacerbate the diagnosis.

### 7.3 Diagnostic Model: Medication Whitelist

Generally, the computer system can access a medication whitelist for a particular diagnosis and defined within a module—of the diagnostic model—corresponding to the particular diagnosis. In particular, the computer system can access a medication whitelist defining a set of whitelisted medications (e.g., one or more whitelisted medications) known to treat and/or mitigate the particular diagnosis. For example, the computer system can implement a diagnostic model including: a first module defining a first diagnosis of thrombosis and a first set of whitelisted medications including anticoagulants configured to thin blood and dissolve blood clots; a second module defining a second diagnosis of diabetes and a second set of whitelisted medications including insulin treatments (e.g., oral, injection); a third module defining a third diagnosis of hyperkalemia and a third set of whitelisted medications including calcium-containing drugs—which may treat effects of elevated potassium levels on the heart and/or muscles—and glucose- and/or insulin-containing injections, which may reduce potassium levels.

### 7.4 Diagnostic Model: Medication Blacklist

Generally, the computer system can access a medication blacklist for a particular diagnosis and defined within a module—of the diagnostic model—corresponding to the particular diagnosis. In particular, the computer system can access a medication blacklist defining a set of blacklisted medications (e.g., one or more blacklisted medications) known and/or predicted to exacerbate the particular diagnosis. For example, the computer system can implement a diagnostic model including: a first module defining a first diagnosis of thrombosis and a first set of blacklisted medications including oral contraceptives, which may increase risk of blood clots in patients diagnosed with thrombosis; a second module defining a second diagnosis of diabetes and a second set of blacklisted medications including corticosteroids, which may increase blood sugar levels and increase insulin resistance; a third module defining a third diagnosis of hyperkalemia and a third set of blacklisted medications including potassium supplements, which may increase potassium levels above a threshold level defined for potassium in patients diagnosed with hyperkalemia.

### 7.4.1 Blacklisted Compounds

In one implementation, the computer system can access a medication blacklist—defined for a particular diagnosis in the diagnostic model—including a list of blacklisted compounds configured to exacerbate the particular diagnosis when included in a medication implemented by a patient diagnosed with the particular diagnosis. In this implementation, rather than generate a list of each medication containing a blacklisted compound, the computer system can: store individual blacklisted compounds in the medication blacklist; scan a list of current medications implemented by a patient diagnosed with the particular diagnosis for medications containing the blacklisted compound; and selectively flag medications, in the list of current medications, containing the particular compound.

In particular, in this implementation, the computer system can: implement a diagnostic model including a first module defining a first diagnosis and a first set of blacklisted compounds configured to exacerbate the first diagnosis when contained in a medication implemented by a patient diagnosed with the first diagnosis; and access a list of current medications implemented by the patient. Then, for each medication, in the list of current medications, the computer system can: access a list of compounds contained in the medication; and scan the list of compounds for blacklisted compounds included in the first set of blacklisted compounds defined for the first diagnosis. In response to a particular medication, in the list of current medications, including a blacklisted compound, in the first set of blacklisted compounds, the computer system can flag this particular medication for review by the patient's provider and/or immediately alert the provider via the provider portal.

For example, in response to a new diagnosis of hyperkalemia for a patient, the computer system can: implement a diagnostic model including a module defining a diagnosis of hyperkalemia and a set of blacklisted compounds—configured to elevate potassium levels above a threshold level in patients diagnosed with hyperkalemia—including potassium and/or potassium-containing compounds; and access a set of current medications prescribed and/or currently implemented by the patient; and, in response to the set of current medications including a first multivitamin of a first type, access a set of compounds contained in multivitamins of the first type. Then, in response to the set of compounds—contained in multivitamins of the first type—including a potassium-containing compound defined in the set of blacklisted compounds, the computer system can: flag the first multivitamin as containing a blacklisted compound configured to exacerbate hyperkalemia; generate a notification—indicating that the first multivitamin contains the blacklisted compound—including a prompt to review implementation of the first multivitamin by the patient and/or to suggest a replacement multivitamin for implementation by the patient; and transmit the notification to the provider via the provider portal.

Additionally or alternatively, in the preceding example, the computer system can: access a list of (common) multivitamins; identify a subset of multivitamins—omitting any blacklisted compound in the set of blacklisted compounds defined for the diagnosis of hyperkalemia—from this list of multivitamins; populate the notification with the subset of multivitamins and a prompt to review the subset of multivitamins and/or suggest a particular multivitamin, from the subset of multivitamins, for implementation by the patient in replacement of the first multivitamin; and transmit the notification to the provider via the provider portal for review. The computer system can therefore automatically suggest replacement medications for the patient—that the provider may further review—responsive to flagging a particular medication implemented by the patient for containing blacklisted compounds.

Therefore, in this implementation, rather than store each individual medication containing a particular compound—known to exacerbate a particular diagnosis—within a module of the diagnostic model corresponding to the particular diagnosis, the computer system can store and access a list of blacklisted compounds and verify whether medications (e.g., currently implemented by a patient) contain these blacklisted compounds. The computer system can then immediately surface any medication—currently implemented by the patient—containing a blacklisted compound defined for a particular diagnosis exhibited by the patient.

### 7.4.2 Blacklist for Each “Whitelisted” Medication

In one implementation, the computer system can populate a medication blacklist with a set of medications configured to elicit a negative response and/or reaction when implemented in combination with a whitelisted medication, in the medication whitelist, for a particular diagnosis.

In particular, in this implementation, for a particular diagnosis, the computer system can populate a medication whitelist with a set of “whitelisted” medications configured to treat the particular diagnosis, such as by eliciting a target response (e.g., a positive response) responsive to implementation by a patient diagnosed with the particular diagnosis. For each whitelisted medication, in the set of whitelisted medications stored in the medication whitelist, the computer system can: initialize a medication blacklist for the whitelisted medication; and populate the medication blacklist with a set of blacklisted medications configured to elicit a negative response or reaction—such as characterized by worsening the particular diagnosis—when implemented (e.g., consumed, applied) in combination with the whitelisted medication.

For example, in response to a new diagnosis for a patient, the computer system can: access the diagnostic model containing the population of modules corresponding to the population of diagnoses; retrieve a first module, in the population of modules, corresponding to the new diagnosis; and extract a medication whitelist—including a set of whitelisted medications configured to treat the new diagnosis—defined in the first module. In particular, in this example, the computer system can access the medication whitelist defining: a first whitelisted medication—including a first set of compounds—configured to treat the diagnosis; and a second whitelisted medication—including a second set of compounds—configured to treat the diagnosis. Then, for the first whitelisted medication, the computer system can access: a first set of blacklisted medications defined for the first whitelisted medication and configured to exacerbate the new diagnosis and/or elicit an unwanted reaction (e.g., a detrimental or harmful response) when implemented (e.g., consumed, applied) in combination with the first whitelisted medication; and a second set of blacklisted medications defined for the second whitelisted medication and configured to exacerbate the new diagnosis and/or elicit an unwanted reaction when implemented in combination with the second whitelisted medication.

The computer system can then access a list of current medications implemented by the patient and scan the first and second set of blacklisted medications for medications included in the list of current medications. In this example, in response to the first set of blacklisted medications including a first medication, in the list of current medications, the computer system can: selectively suggest the second whitelisted medication over the first whitelisted medication; notify the provider of the unwanted reaction between the first medication and the first whitelisted medication for the new diagnosis; and/or flag the unwanted interaction between the first medication and the first whitelisted medication and notify the provider to review before prescribing the first whitelisted medication as treatment for the new diagnosis.

### 7.5 Medication Report

Generally, the computer system can scan a list of current medications implemented by the patient for medications included in the medication whitelist and/or medication blacklist defined for a particular diagnosis associated with the patient. The computer system can then generate a medication report—including a list of whitelisted and/or blacklisted medications currently implemented by the patient—for review by the provider. The computer system can thus immediately surface any medications implemented by the patient that may treat and/or exacerbate the particular diagnosis.

In particular, in response to a new diagnosis for a patient, the computer system can: access a module, in the population of modules defined in the diagnostic model, corresponding to the new diagnosis; extract a medication whitelist—including a list of whitelisted medications configured to treat the new diagnosis—defined by the module; extract a medication blacklist—including a list of blacklisted medications configured to exacerbate the new diagnosis—defined by the module; and access a list of current medications implemented by the patient. The computer system can then: scan the medication whitelist for medications included in the list of current medications; scan the medication blacklist for medications included in the list of current medications; in response to the medication whitelist including a first medication included in the list of current medications, populate a medication report—associated with the new diagnosis—with the first medication and indicate approval of the first medication for treating the first diagnosis; and, in response to the medication blacklist including a second medication included in the list of current medications, populate the medication report with the second medication and indicate association of the second mediation with exacerbation of the first diagnosis. Furthermore, the computer system can populate the medication report with a prompt to review implementation of the second medication by the patient. The computer system can then present this medication report to the provider via the patient portal, such as in near real-time responsive to receiving and/or predicting the new diagnosis.

In one example, the computer system can execute the methods and techniques described above to predict a thrombosis diagnosis for a patient. The computer system can then: access a medication whitelist—defined in the thrombosis module—including anticoagulants configured to thin blood and dissolve blood clots to treat the thrombosis diagnosis; and, in response to the list of current medications including an anticoagulant, in the set of whitelisted medications, flag the anticoagulant for review by the provider (e.g., via the provider portal). Therefore, the computer system can flag a particular medication—from the list of current medications—known to treat and/or mitigate the particular diagnosis for review by the provider.

In another example, the computer system can execute the methods and techniques described above to predict a heart failure diagnosis for a patient. The computer system can then: access a medication blacklist including a set of blacklisted compounds predicted to exacerbate the diagnosis including calcium channel blockers; and, in response to a list of current medications implemented by the patient including a calcium channel blocker, in the set of blacklisted compounds, flag the calcium channel blocker for review by the provider (e.g., via the provider portal). Therefore, the computer system can flag a particular compound—from the list of current medications—known to exacerbate the particular diagnosis when included in a medication implemented by a patient diagnosed with the particular diagnosis.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for augmenting medical documentation comprising:
for a first encounter with a first patient, receiving a first diagnosis for the first patient from a provider via a provider portal executing on a computing device accessed by the provider;
accessing a health record, in a population of health records, corresponding to the first patient and comprising a corpus of patient data associated with the first patient;
accessing a diagnostic model comprising a population of modules corresponding to a population of diagnoses, each module, in the population of modules, defining a set of target indicators supporting a corresponding diagnosis in the population of diagnoses;
accessing a first set of target indicators defined for the first diagnosis in a first module, in the population of modules, corresponding to the first diagnosis;
extracting a first set of patient indicators, from the corpus of patient data, corresponding to the first set of target indicators and supporting the first diagnosis for the first patient;
generating a first notification comprising the first set of patient indicators and a first prompt to review each patient indicator, in the first set of patient indicators, for specifying in a first provider note generated for the first encounter and indicating the first diagnosis for the first patient;
transmitting the first notification to the provider via the provider portal; and
in response to receiving selection of a first subset of patient indicators, in the first set of patient indicators, from the provider via the provider portal:
appending the first provider note with the first subset of patient indicators linked to the first diagnosis;
predicting a first acceptance score for the first provider note based on the first diagnosis and the first subset of patient indicators, the first acceptance score representing a likelihood of acceptance of the first diagnosis for the first patient during the first encounter represented by the first provider note; and
in response to the first acceptance score exceeding a threshold score, verifying the first provider note.

2. The method of claim 1:
wherein accessing the first set of target indicators comprises accessing:
a set of primary target indicators supporting the first diagnosis; and
a set of secondary target indicators supporting the first diagnosis in combination with the set of primary target indicators; and
wherein generating the first notification comprising the first set of target indicators comprises:
accessing a first quantity of patient indicators, in the first set of patient indicators, corresponding to the set of primary target indicators;
accessing a second quantity of patient indicators, in the first set of patient indicators, corresponding to the set of secondary target indicators;
in response to the first quantity exceeding a threshold quantity, populating the first notification with the first quantity of patient indicators corresponding to the set of primary target indicators; and
in response to the first quantity falling below the threshold quantity:
populating the first notification with the first quantity of patient indicators corresponding to the set of primary target indicators; and
populating the first notification with a subset of the second quantity of patient indicators corresponding to the set of secondary target indicators.

3. The method of claim 1, further comprising, in response to the first acceptance score falling below the threshold score:
withholding verification of the first provider note;
generating a second notification indicating the first acceptance score falling below the threshold score and comprising a prompt to select additional patient indicators, in the first set of patient indicators, for specifying in the first provider note; and
transmitting the second notification to the provider via the provider portal.

4. The method of claim 3, further comprising, in response to transmitting the second notification to the provider and in response to receiving selection of a second subset of patient indicators, in the first set of patient indicators:
appending the first provider note with the second subset of patient indicators;
predicting a second acceptance score for the first provider note based on the first diagnosis, the first subset of patient indicators, and the second subset of patient indicators; and
in response to the second acceptance score exceeding the threshold score, verifying the first provider note.

5. The method of claim 1:
wherein accessing the diagnostic model comprising the population of modules comprises accessing the diagnostic model comprising the population of modules, each module, in the population of modules, defining:
the set of target indicators supporting the corresponding diagnosis in the population of diagnoses; and
a set of treatment pathways predicted to treat the corresponding diagnosis;
further comprising:
accessing a first set of treatment pathways defined for the first diagnosis in the first module and predicted to treat the first diagnosis; and
identifying a first treatment pathway, in the first set of treatment pathways, selected for the patient and specified in the first provider note; and
wherein predicting the first acceptance score for the first provider note based on the first diagnosis and the first subset of patient indicators comprises predicting the first acceptance score for the first provider note based on the first diagnosis, the first subset of patient indicators, and the first treatment pathway.

6. The method of claim 5:
wherein appending the first provider note with the first subset of patient indicators linked to the first diagnosis comprises:
initializing the first provider note for the first encounter with the first patient; and
appending the first provider note with the first diagnosis, the first set of patient indicators supporting the first diagnosis, and the first treatment pathway; and
wherein verifying the first provider note comprises. in response to the first acceptance score exceeding the threshold score:
generating a second notification comprising a second prompt to verify completion of the first provider note;
transmitting the second notification to the provider via the provider portal; and
in response to receiving verification of the first provider note from the provider, verifying the first provider note for transmitting to an insurance company affiliated with the first patient.

7. The method of claim 1, further comprising, in response to receiving selection of the first subset of patient indicators:
generating a data packet comprising:
the first diagnosis;
the first subset of patient indicators selected by the provider; and
a second subset of patient indicators, in the first set of patient indicators, unselected by the provider;
labelling the first subset of patient indicators, stored in the data packet, with a first value representing selection of the first subset of patient indicators by the provider;
labelling the second subset of patient indicators, stored in the data packet, with a second value representing rejection of the second subset of patient indicators by the provider; and
storing the data packet in a library of data packets linked to a provider profile, in a population of provider profiles, associated with the provider.

8. The method of claim 7, further comprising:
for a second encounter with a second patient, receiving the first diagnosis for the second patient from the provider via the provider portal;
accessing a second health record, in the population of health records, corresponding to the second patient and comprising a second corpus of patient data associated with the second patient;
extracting a second set of patient indicators, from the second corpus of patient data, corresponding to the first set of target indicators and supporting the first diagnosis for the second patient;
based on the library of data packets linked to the provider profile, selecting a second subset of patient indicators, in the second set of patient indicators, for presenting to the provider;
populating a second notification with the second subset of patient indicators and a second prompt to select patient indicators, from the second subset of patient indicators, for specifying in a second provider note generated for the second encounter; and
transmitting the second notification to the provider via the provider portal.

9. The method of claim 1, further comprising:
generating a data packet corresponding to the provider note;
recording the first diagnosis and the first subset of patient indicators specified in the provider note to the data packet;
receiving confirmation of acceptance of the provider note by an insurance company affiliated with the first patient;
labeling the data packet as accepted; and
storing the data packet in a library of data packets stored in an insurer profile, in a population of insurer profiles, affiliated with the insurance company.

10. The method of claim 1:
wherein accessing the first set of target indicators comprises accessing a first target indicator and a second target indicator;
wherein extracting the first set of patient indicators comprises extracting a first patient indicator corresponding to the first target indicator; and
further comprising, in response to detecting absence of a second patient indicator corresponding to the second target indicator in the corpus of patient data:
accessing a target test method defined for the second target indicator in the first module, the target test method configured to yield patient data corresponding to the second target indicator;
generating a second notification indicating insufficient evidence in the health record to support the first diagnosis and comprising a suggestion to execute the target test method for the first patient; and
transmitting the second notification to the provider via the provider portal.

11. The method of claim 1, further comprising:
at a first time, in response to receiving selection of a second subset of patient indicators, in the first set of patient indicators, for specifying in a secondary note:
recording the second subset of patient indicators in the secondary note; and
linking the secondary note, associated with the first provider note, to the health record of the first patient;
at a second time succeeding the first time, in response to receiving confirmation of rejection of the first provider note by an entity associated with the patient and different from the provider:
accessing the secondary note;
generating a second notification comprising a second prompt to select additional indicators from the second subset of patient indicators for recording in the first provider note; and
transmitting the second notification to the provider via the provider portal; and
in response to receiving selection of a third subset of patient indicators in the second subset of patient indicators, appending the first provider note with the third subset of patient indicators.

12. The method of claim 1:
wherein receiving the first diagnosis comprises receiving a primary diagnosis as the first diagnosis;
wherein accessing the diagnostic model comprises accessing the first module corresponding to the primary diagnosis of the first diagnosis and comprising:
a first submodule corresponding to a first subdiagnosis of the primary diagnosis, the first submodule defining a first subset of secondary target indicators, in the first set of target indicators, supporting the first subdiagnosis; and
a second submodule corresponding to a second subdiagnosis of the primary diagnosis, the second submodule defining a second subset of secondary target indicators, in the first set of target indicators, supporting the second subdiagnosis;
wherein extracting the first set of patient indicators comprises extracting:
a first subset of secondary patient indicators supporting the first subdiagnosis of the primary diagnosis; and
a second subset of secondary patient indicators supporting the second subdiagnosis of the primary diagnosis; and
further comprising:
detecting a first quantity of patient indicators in the first subset of secondary patient indicators and a second quantity of patient indicators in the second subset of secondary patient indicators;
in response to the first quantity of patient indicators in the first subset of secondary patient indicators exceeding a first threshold quantity:
generating a second notification suggesting the first subdiagnosis; and
transmitting the second notification to the provider via the provider portal; and
in response to the second quantity of patient indicators in the second subset of secondary patient indicators exceeding a second threshold quantity:
generating a third notification suggesting the second subdiagnosis; and
transmitting the second notification to the provider via the provider portal.

13. The method of claim 12:
wherein receiving the first diagnosis comprises receiving a heart failure diagnosis as the primary diagnosis;
wherein accessing the first module comprises accessing the first module, corresponding to the heart failure diagnosis, comprising:
the first submodule, corresponding to a systolic heart failure diagnosis as the first subdiagnosis of the heart failure diagnosis, the first subset of secondary target indicators comprising a first target heart rate and a first target blood pressure supporting the systolic heart failure diagnosis; and
the second submodule, corresponding to a diastolic heart failure diagnosis as the second subdiagnosis of the heart failure diagnosis, the second subset of secondary target indicators comprising a second target heart rate and a second target blood pressure supporting the diastolic heart failure diagnosis;
wherein extracting the first set of patient indicators comprises extracting:
the first subset of secondary patient indicators supporting the systolic heart failure diagnosis; and
the second subset of secondary patient indicators supporting the diastolic heart failure diagnosis;
wherein generating the second notification suggesting the first subdiagnosis comprises generating the second notification suggesting the systolic heart failure diagnosis in response to the first quantity of patient indicators in the first subset of secondary patient indicators exceeding the first threshold quantity; and
wherein generating the third notification suggesting the second subdiagnosis comprises generating the third notification suggesting the diastolic heart failure diagnosis in response to the second quantity of patient indicators in the second subset of secondary patient indicators exceeding the second threshold quantity.

14. The method of claim 1, wherein predicting the first acceptance score comprises:
accessing a quantity of patient indicators in the first subset of patient indicators selected by the provider;
characterizing presence of a treatment pathway, in the set of treatment pathways defined in the first module, included in the provider note; and
deriving the first acceptance score based on the quantity of patient indicators and presence of the treatment pathway.

15. The method of claim 14, wherein deriving the first acceptance score based on the quantity of patient indicators and presence of the treatment pathway further comprises:
accessing a threshold quantity of patient indicators defined for the insurance company for the first diagnosis;
characterizing a first difference between the quantity of patient indicators and the threshold quantity;
accessing a set of preferred target indicators defined for the insurance company;
characterizing a second difference between the first subset of patient indicators and the set of preferred patient indicators; and
deriving the first acceptance score based on the first difference, the second difference, and presence of the treatment pathway.

16. A method for augmenting medical documentation comprising:
for an encounter with a patient, receiving a first diagnosis for the patient from a provider via a provider portal executing on a computing device accessed by the provider;
accessing a health record, in a population of health records, corresponding to the patient and comprising a corpus of patient data associated with the patient;
accessing a diagnostic model comprising a population of modules, corresponding to a population of diagnoses, each module, in the population of modules, defining a set of target indicators supporting a corresponding diagnosis in the population of diagnoses;
accessing a first set of target indicators defined for the first diagnosis in a first module, in the population of modules, and supporting the first diagnosis, the first set of target indicators comprising:
a set of primary target indicators supporting the first diagnosis; and
a set of secondary target indicators supporting the first diagnosis in combination with the set of primary target indicators;
extracting a set of primary patient indicators, from the corpus of patient data, corresponding to the set of primary target indicators and supporting the first diagnosis for the patient;
detecting a first quantity of patient indicators in the set of primary patient indicators;
in response to a first quantity of primary patient indicators in the set of primary patient indicators exceeding a threshold quantity:
populating a first notification with the set of primary patient indicators and a first prompt to select indicators, in the set of primary patient indicators, for specifying in a provider note generated for the encounter and indicating the first diagnosis for the patient; and
transmitting the first notification to the provider via the provider portal; and
in response to receiving selection of a first subset of primary patient indicators, in the set of primary patient indicators, appending the provider note with the first subset of primary patient indicators linked to the first diagnosis.

17. The method of claim 16, further comprising:
in response to the first quantity of primary patient indicators falling below the threshold quantity:
extracting a set of secondary patient indicators, from the corpus of patient data, corresponding to the set of secondary target indicators and supporting the first diagnosis in combination with the set of primary patient indicators; and
populating the first notification with the set of primary patient indicators, the set of secondary patient indicators, and the first prompt to select patient indicators, in the set of primary patient indicators and the set of secondary patient indicators, for specifying in the provider note; and
in response to receiving selection of a subset of secondary patient indicators, in the set of secondary patient indicators, appending the provider note with the subset of secondary patient indicators linked to the first diagnosis.

18. The method of claim 16, further comprising, in response to receiving selection of the first subset of primary patient indicators, in the set of primary patient indicators, from the provider via the provider portal:
predicting an acceptance score for the provider note based on the first diagnosis and the first subset of primary patient indicators, the first acceptance score representing a likelihood of acceptance of the first diagnosis for the patient during the encounter represented by the provider note; and
in response to the acceptance score exceeding a threshold score, verifying the provider note for transmittal to an insurance company affiliated with the patient.

19. The method of claim 16:
wherein accessing the first set of target indicators comprises accessing the first set of target indicators, each target indicator, in the set of target indicators, defining a rank, in a set of ranks, based on a correlation between the target indicator and presence of the diagnosis; and
wherein populating the first notification with the set of primary patient indicators comprises:
for each primary patient indicator, in the set of primary patient indicators, inserting the primary patient indicator into a ranked list according to rank of a corresponding target indicator in the first set of target indicators; and
populating the first notification with the ranked list of primary patient indicators.

20. A method for augmenting medical documentation comprising:
for an encounter with a patient, receiving a diagnosis for the patient from a provider via a provider portal executing on a computing device accessed by the provider;
accessing a health record, in a population of health records, corresponding to the patient and comprising a corpus of patient data associated with the patient;
accessing a diagnostic model comprising a population of modules, corresponding to a population of diagnoses, each module, in the population of modules, defining a set of target indicators supporting a corresponding diagnosis in the population of diagnoses;
accessing a set of target indicators defined for the diagnosis in a first module, in the population of modules, corresponding to the diagnosis;
extracting a set of patient indicators, from the corpus of patient data, corresponding to the set of target indicators and supporting the diagnosis for the patient;
selecting a first subset of patient indicators, in the set of patient indicators, for presenting to the provider in support of the diagnosis, the first subset of patient indicators predicted to yield a threshold probability of acceptance of a provider note specifying the diagnosis and the first subset of patient indicators;
generating a notification comprising the first subset of patient indicators and a prompt to review each patient indicator, in the first subset of patient indicators, for specifying in the provider note generated for the encounter and indicating the diagnosis for the patient;
transmitting the notification to the provider via the provider portal; and
in response to receiving selection of a second subset of patient indicators, in the first subset of patient indicators, from the provider via the provider portal, appending the provider note with the second subset of patient indicators linked to the diagnosis.
